SEQUENCE LISTING (57 sequences)

SEQ ID NO: 1 - protein, 146 aa, Homo sapiens
VHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 2 - protein, 10 aa, synthetic construct
GRISSIIHMA

SEQ ID NO: 3 - protein, 13 aa, synthetic construct
RVGTTVYADSVKG

SEQ ID NO: 4 - protein, 11 aa, synthetic construct
LQYEKHGGADY

SEQ ID NO: 5 - protein, 5 aa, synthetic construct
GGGGE

SEQ ID NO: 6 - protein, 256 aa, synthetic construct
QVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGEGGGGEGGGGEVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 7 - protein, 10 aa, synthetic construct
GFSLTTYGVH

SEQ ID NO: 8 - protein, 16 aa, synthetic construct
VIWSGGDTDYNASFIS

SEQ ID NO: 9 - protein, 14 aa, synthetic construct
NKDYYTNYDFTMDY

SEQ ID NO: 10 - protein, 17 aa, synthetic construct
KSSQSVLYSSNQKNFLA

SEQ ID NO: 11 - protein, 7 aa, synthetic construct
WASTRES

SEQ ID NO: 12 - protein, 8 aa, synthetic construct
HQYLSSYT

SEQ ID NO: 13 - protein, 10 aa, synthetic construct
GYTFIDYWIE

SEQ ID NO: 14 - protein, 16 aa, synthetic construct
EIFPGSGTINHNEKFK

SEQ ID NO: 15 - protein, 5 aa, synthetic construct
EGLDY

SEQ ID NO: 16 - protein, 10 aa, synthetic construct
SASSSVSYIY

SEQ ID NO: 17 - protein, 7 aa, synthetic construct
DTSTLAS

SEQ ID NO: 18 - protein, 9 aa, synthetic construct
QQWSRNPFT

SEQ ID NO: 19 - protein, 10 aa, synthetic construct
GFSLTSYGVH

SEQ ID NO: 20 - protein, 16 aa, synthetic construct
VIWSGGSTDYNAAFIS

SEQ ID NO: 21 - protein, 14 aa, synthetic construct
NKDFYSNYDYTMDY

SEQ ID NO: 22 - protein, 17 aa, synthetic construct
KSSQSVLYSSNQKNFLA

SEQ ID NO: 23 - protein, 7 aa, synthetic construct
WASTRES

SEQ ID NO: 24 - protein, 8 aa, synthetic construct
HQYLSSYT

SEQ ID NO: 25 - protein, 10 aa, synthetic construct
  VARIANT 4: note = X is any naturally occurring amino acid
GYTXTAYGIN

SEQ ID NO: 26 - protein, 17 aa, synthetic construct
YIYIGNGYTDYNEKFKG

SEQ ID NO: 27 - protein, 11 aa, synthetic construct
SGWDEDYAMDF

SEQ ID NO: 28 - protein, 11 aa, synthetic construct
RASENIYSYLA

SEQ ID NO: 29 - protein, 7 aa, synthetic construct
HAKTLAE

SEQ ID NO: 30 - protein, 9 aa, synthetic construct
QHHYGPPPT

SEQ ID NO: 31 - protein, 119 aa, synthetic construct
QVQVVESGGGLRQTGGSLRLSCTASGRIFEVNMMAWYRQAPGKQRELVAEISRVGTTVYADSVKGRFTISRDSAKNTVTLQMNSLKSEDTAVYYCNALQYDRYGGAEYWGQGTQVTVSS

SEQ ID NO: 32 - protein, 119 aa, synthetic construct
QVQLAESGGGLVQAGDSLKLSCTASGRIFEVNMMAWYRQAPGKDRELVAEISRVGTTTYADSVKGRFTISRDSAKNTVTLQMNSLKSEDTAVYYCNALQYSRYGGAEYWGQGTQVTVSG

SEQ ID NO: 33 - protein, 119 aa, synthetic construct
QVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSG

SEQ ID NO: 34 - protein, 119 aa, synthetic construct
QVQLVESGGGLRQTGESLRLSCTASGRIFEVNMMAWYRQAPGKQRELVAEISRVGTTTYADSVKGRFTISRDSAKNTVTLQMNSLKSEDTAVYYCNALQYDRYGGAEYWGQGTQVTVSG

SEQ ID NO: 35 - protein, 258 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSLEVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSS

SEQ ID NO: 36 - protein, 257 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 37 - protein, 256 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGDGGGGDGGGGEVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSS

SEQ ID NO: 38 - protein, 256 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGEGGGGEGGGGEVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSS

SEQ ID NO: 39 - protein, 257 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRISSIIHMAWVRQAPGKQRELVSEISRVGTTVYADSVKGRFTISRDNSKNTLYLQMNSLRAEDTAVYYCNALQYEKHGGADYWGQGTLVTVSS

SEQ ID NO: 40 - protein, 256 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGDGGGGDGGGGEVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 41 - protein, 256 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGEGGGGEGGGGEVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 42 - protein, 256 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGDGGGGDGGGGEVQLVESGGGLVQPGGSLRLSCAASGRISSIIHMAWVRQAPGKQRELVSEISRVGTTVYADSVKGRFTISRDNSKNTLYLQMNSLRAEDTAVYYCNALQYEKHGGADYWGQGTLVTVSS

SEQ ID NO: 43 - protein, 140 aa, synthetic construct
MAVLGLLFCLVTFPSCVLSQVQLKQSGPGLVQPSQSLSISCTVSGFSLTTYGVHWVRQSPGKGLEWLGVIWSGGDTDYNASFISRLRINKDTSKSQVFFKMNSLQADDTAYYCARNKDYYTNYDFTMDYWGQGTSVTVSS

SEQ ID NO: 44 - protein, 132 aa, synthetic construct
MESQTQVFLSLLLWVSGTCGNIMMTQSPSSLAVSAGEKVTMSCKSSQSVLYSSNQKNFLAWYQQKPGQSPKLLIYWASTRESGVPDRFTGSGSGTDFILTINSVQVEDQAVYYCHQYLSSYTFGGGTKLEIK

SEQ ID NO: 45 - protein, 133 aa, synthetic construct
MDWTWVFLFLLSVTAGVHSQVQLLQSGAEVMKPGASVTLSCKAIGYTFIDYWIEWIKQRPGHGLEWIGEIFPGSGTINHNEKFKDKASFSAHSSSNTAYMQLSRLTSEDSAIYYCAREGLDYWGQGTTLTVSS

SEQ ID NO: 46 - protein, 128 aa, synthetic construct
MDFQVQIFSFLLISASVIISRGQIVLTQSPAIMSASPGERVTMTCSASSSVSYIYWYQQKSGTSPKRWIFDTSTLASGVPTRFSGSGSGTSYSLTISSMETEDAATYYCQQWSRNPFTFGSGTKLEIK

SEQ ID NO: 47 - protein, 141 aa, synthetic construct
MAVLGLLFCLVTFPSCVLSQVQLKQSGPGLVQPSQSLSITCTVSGFSLTSYGVHWVRQSPGKGLEWLGVIWSGGSTDYNAAFISRLSISKDTSKSQVFFKMNSLQPDDTAIYYCARNKDFYSNYDYTMDYWGQGTSVTVSS

SEQ ID NO: 48 - protein, 132 aa, synthetic construct
MESQTQVFLSLLLWVSGTCGNIMMTQSPSFLAVSAGEKVTLSCKSSQSVLYSSNQKNFLAWYQQKPGQSPKLLIYWASTRESGVPDRFTGSGSGTDFTLTISSVQAEDLAVYYCHQYLSSYTFGGGTKLEIK

SEQ ID NO: 49 - protein, 141 aa, synthetic construct
MAVLGLLFCLVTFPSCVLSEVQLVESGGGLVQPGGSLRLSCAASGFSLTTYGVHWVRQAPGKGLEWVSVIWSGGDTDYNASFISRFTISRDNSKNTLYLQMNSLRAEDTAVYYCARNKDYYTNYDFTMDYWGQGTTVTVSS

SEQ ID NO: 50 - protein, 132 aa, synthetic construct
MESQTQVFLSLLLWVSGTCGDIVMTQSPDSLAVSLGERATINCKSSQSVLYSSNQKNFLAWYQQKPGQPPKLLIYWASTRESGVPDRFSGSGSGTDFTLTISSLQAEDVAVYYCHQYLSSYTFGQGTKLEIK

SEQ ID NO: 51 - protein, 133 aa, synthetic construct
MDWTWVFLFLLSVTAGVHSQVQLVQSGAEVKKPGSSVKVSCKASGYTFIDYWIEWVRQAPGQGLEWMGEIFPGSGTINHNEKFKDRVTITADKSTSTAYMELSSLRSEDTAVYYCAREGLDYWGQGTLVTVSS

SEQ ID NO: 52 - protein, 126 aa, synthetic construct
MEAPAQLLFLLLLWLPDTTGEIVLTQSPATLSLSPGERATLSCSASSSVSYIYWYQQKPGQAPRLLIYDTSTLASGIPARFSGSGSGTDFTLTISSLEPEDFAVYYCQQWSRNPFTFGPGTKVDIK

SEQ ID NO: 53 - protein, 106 aa, synthetic construct
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - DNA, 774 bp, synthetic construct
  source 1..774: mol_type = other DNA
caggtgcagctggtggaaagcggcggaggcctggtcaagcctggcggcagcctgagactgagctgtgccgccagcggcagacccgtgtccaattacgccgctgcctggttccggcaggcccctggcaaagagagagagttcgtcagcgccatcaactggcagaaaaccgccacctacgccgacagcgtgaagggccggttcaccatcagccgggacaacgccaagaacagcctgtacctgcagatgaactccctgcgggccgaggacaccgccgtgtactactgcgccgctgtgttccgggtggtggcccccaagacccagtacgactacgattactggggccagggcaccctggtcaccgtgtcatctggcggagggggagaaggcgggggaggggaagggggaggcggcgaagtccagctgctggaatctgggggcggactggtgcagccaggcggctccctcagactgtcttgcgccgcctccggccggatcagcagcatcatccacatggcctggtttagacaggctcccggaaaagaacgcgagctggtgtccgagatctccagagtgggcaccaccgtgtatgccgactccgtgaaaggcagattcacaatctcccgcgacaacagcaagaatactctgtatctccagatgaatagcctgaagcccgaagatacagccgtctactattgcaacgccctgcagtacgagaagcacggcggagccgactattggggacagggaacactcgtgacagtgtctagctgatga

SEQ ID NO: 55 - protein, 8 aa, synthetic construct
GRPVSNYA

SEQ ID NO: 56 - protein, 8 aa, synthetic construct
INWQKTAT

SEQ ID NO: 57 - protein, 17 aa, synthetic construct
AAVFRVVAPKTQYDYDY

(19) United States
(12) Patent Application Publication
(10) Pub. No.: US 2024/0343787 A1
(43) Pub. Date: Oct. 17, 2024

(54) METHODS FOR TREATING SICKLE CELL DISEASE OR BETA THALASSEMIA USING COMPLEMENT ALTERNATIVE PATHWAY INHIBITORS

(71) Applicant: Alexion Pharmaceuticals, Inc., Boston, MA (US)

(72) Inventors: Sung-Kwon Kim, Cheshire, CT (US); Stephanie L. Beq, Le Kremlin-Bicetre (FR)

(73) Assignee: Alexion Pharmaceuticals, Inc., Boston, MA (US)

(21) Appl. No.: 18/682,386

(22) PCT Filed: Aug. 18, 2022
(86) PCT No.: PCT/US2022/040720

§ 371 Date: Feb. 8, 2024

Related U.S. Application Data
(60) Provisional application No. 63/235,320, filed on Aug. 20, 2021.
(60) Provisional application No. 63/349,270, filed on Jun. 6, 2022.

(51) Int. Cl. C07K 16/18; A61P 7/06; C12N 15/113

(52) U.S. Cl. CPC C07K 16/18; A61P 7/06; C12N 15/113; C07K 2317/31; C12N 2310/11; C12N 2310/14; C12N 2310/141; C12N 2310/531

(57) ABSTRACT

The present invention relates to methods, uses, and compositions for the treatment of Sickle cell disease (SCD), beta thalassemia (BT), sickle cell BT. More specifically, the invention concerns the treatment of patients having SCD, BT, or sickle cell BT using a complement pathway component (e.g., Factor P (properdin)) inhibitor, such as an antibody or fragment thereof, a nucleic acid molecule, a peptide, a small molecule, or an aptamer, among others.

### BACKGROUND

Sickle cell disease (SCD) is the most common monogenic disease worldwide. In some forms, the disease is caused by mutations in the β globin gene, e.g., a single nucleotide mutation in β globin gene resulting in glutamic acid substitution by valine at position 6, the gene that is also responsible for causing beta thalassemia (BT) and sickle cell BT. Despite extensive recognition of the underlying cause of the disease, few treatment options are available to control SCD symptoms. Two main manifestations of SCD, anemia and vaso-occlusion crisis (VOC), affects the mortality, morbidity and quality of life for SCD patients. Although there are two approved treatment options, hydroxyurea and L-glutamine, for SCD patients, they are generally considered suboptimal attenuating disease symptoms. Accordingly, there is a need in the art for treating such conditions.

### SUMMARY

Described herein are compositions that specifically or substantially specifically bind to a complement pathway component (e.g., Factor P (properdin)) and selectively block alternative complement pathway activation. By inhibiting the functional activity of the alternative complement pathway, e.g., by inhibiting properdin, the alternative complement pathway inhibitors (e.g., an anti-Factor P monovalent antibody or fragment thereof) described herein inhibits alternative complement pathway-induced assembly of the membrane attack complex. In addition, selective binding of a single properdin molecule with a properdin inhibitor can reduce undesirable immune complexes, resulting from aggregation. Thus, the selective targeting of properdin, e.g., properdin monomer or multimer, can, in turn, improve clinical benefits for patients with sickle cell disease (SCD), beta thalassemia (BT), or sickle cell BT.

The instant disclosure is based, in part, on the discovery that inhibitors of alternative complement pathway, such as, e.g., Factor P (properdin) inhibitors, can attenuate and even halt symptoms of SCD. By using an established laboratory model for SCD (Townes SS mice subjected to hypoxic conditions), the instant disclosure demonstrates, for the first time, that treating animals with anti-properdin inhibitors inhibited the pathophysiology of SCD, vis-à-vis: (1) inhibition of complement deposition on red blood cells (RBC); (2) attenuation of intravascular hemolysis; and/or reduction in the severity of VOC. More specifically, using an established cellular model, the disclosure shows that enhanced complement fragment deposition of C5b9 and C3 in RBCs of the SCD mice under hypoxic conditions was reversed via pretreatment with anti-properdin monoclonal antibody (MAb). Additionally, increases in the level of intravascular hemolysis under hypoxic conditions (as measured by plasma lactate dehydrogenase (LDH) activity, free heme and free hemoglobin, and/or total bilirubin levels), was effectively attenuated by pretreatment with anti-properdin MAb. Third, increased vaso-occlusion in the vessels of vital organs such as lung and liver of SCD mice under hypoxic conditions was effectively reduced by pretreatment with anti-properdin MAb, which effect was not observed in sham (control) SCD mice pretreated with buffer. These data establish that an anti-complement antibody, such as an anti-properdin antibody, protects SCD animals from injury at both the cellular and organ level. The scientific evidence provided by the instant disclosure supports use of complement inhibitors, especially, properdin antagonists such as anti-properdin antibodies, in the treatment of SCD and related conditions such as BT and sickle BT.

In one aspect, the disclosure features a method for treating SCD in a subject, including administering to the subject an effective amount of a composition including a complement alternative pathway inhibitor.

In another aspect, the disclosure features a method for treating BT in a subject, including administering to the subject an effective amount of a composition including a complement alternative pathway inhibitor.

In another aspect, the disclosure features a method for treating sickle cell BT in a subject, including administering to the subject an effective amount of a composition including a complement alternative pathway inhibitor.

In some embodiments of any of the foregoing aspects, the complement alternative pathway inhibitor is selected from the group consisting of an antibody or an antigen-binding fragment thereof, a peptide, a small molecule, a nucleic acid molecule, and an aptamer.

In some embodiments of any of the foregoing aspects, the complement alternative pathway inhibitor is a properdin inhibitor.

In some embodiments of any of the foregoing aspects, the properdin inhibitor is an anti-properdin antibody or antigen-binding fragment thereof.

In some embodiments of any of the foregoing aspects, the anti-properdin antibody or antigen-binding fragment thereof includes:
CDR-H1 (SEQ ID NO: 2), CDR-H2 (SEQ ID NO: 3), and CDR-H3 (SEQ ID NO: 4).

In some embodiments of any of the foregoing aspects, the anti-properdin antibody or antigen binding fragment thereof includes: CDR-H1 (SEQ ID NO: 7), CDR-H2 (SEQ ID NO: 8), CDR-H3 (SEQ ID NO: 9), CDR-L1 (SEQ ID NO: 10), CDR-L2 (SEQ ID NO: 11), and CDR-L3 (SEQ ID NO: 12); CDR-H1 (SEQ ID NO: 13), CDR-H2 (SEQ ID NO: 14), CDR-H3 (SEQ ID NO: 15), CDR-L1 (SEQ ID NO: 16), CDR-L2 (SEQ ID NO: 17), and CDR-L3 (SEQ ID NO: 18); CDR-H1 (SEQ ID NO: 19), CDR-H2 (SEQ ID NO: 20), CDR-H3 (SEQ ID NO: 21), CDR-L1 (SEQ ID NO: 22), CDR-L2 (SEQ ID NO: 23), and CDR-L3 (SEQ ID NO: 24); or CDR-H1 (SEQ ID NO: 25), CDR-H2 (SEQ ID NO: 26), CDR-H3 (SEQ ID NO: 27), CDR-L1 (SEQ ID NO: 29), CDR-L2 (SEQ ID NO: 29), and CDR-L3 (SEQ ID NO: 30).

In some embodiments of any of the foregoing aspects, the anti-properdin antibody includes: the heavy chain (HC) of SEQ ID NO: 43 and the light chain (LC) of SEQ ID NO: 44; the HC of SEQ ID NO: 45 and the LC of SEQ ID NO: 46; the HC of SEQ ID NO: 47 and the LC of SEQ ID NO: 48; the HC of SEQ ID NO: 49 and the LC of SEQ ID NO: 50; the HC of SEQ ID NO: 51 and the LC of SEQ ID NO: 52; or the HC of SEQ ID NO: 53 and the LC of SEQ ID NO: 44.

In some embodiments of any of the foregoing aspects, the anti-properdin antibody or antigen-binding fragment thereof includes: the anti-FP VHH component of SEQ ID NO: 6; the sequence of SEQ ID NO:6; the V_{HH} of SEQ ID NO: 31; the V_{HH} of SEQ ID NO: 32; the V_{HH} of SEQ ID NO: 33; or the V_{HH} of SEQ ID NO: 34.

In some embodiments of any of the foregoing aspects, the peptide inhibits complement factor C3.

In some embodiments of any of the foregoing aspects, the small molecule is a complement factor D inhibitor.

In some embodiments of any of the foregoing aspects, the composition includes the complement inhibitor and a pharmaceutically acceptable carrier.

In some embodiments of any of the foregoing aspects, the method reduces intravascular hemolysis in the subject.

In some embodiments of the foregoing aspects, the SCD includes hemolytic anemia or an acute VOC event. In some embodiments, the VOC event is a lung VOC and/or a liver VOC. For example, in some embodiments, the lung VOC manifests as acute chest syndrome (ACS) and/or chronic lung disease; and/or the liver VOC manifests as severe abdominal pain and/or liver dysfunction.

In some embodiments of any of the foregoing aspects, the subject presents with abdominal meteorism, right upper quadrant pain, or acute painful hepatomegaly.

In some embodiments of any of the foregoing aspects, the subject is a human patient diagnosed as having SCD, BT, or sickle cell BT.

In some embodiments of any of the foregoing aspects, the human patient is under 18 years of age.

In some embodiments of the foregoing aspects, the subject having SCD is diagnosed as having a mutation in the β globin gene. For example, in some embodiments, the mutation in the β globin gene is a single nucleotide mutation in the β globin gene. In some embodiments, the single nucleotide mutation in the β globin gene results in a glutamic acid substitution by valine at position 6, relative to SEQ ID NO: 1: VHLTPEEKSAVTALWGKVNVDEVGGEALGRLLVVYPWTQRFFESFGDLSTPDAVMGNPKVKAHGKKVL GAFSDGLAHLDNLKGTFATLSELHCDKLHVDPENFRLLGNVLVCVLAHHFGKEFTPPVQAAYQKVVAGV ANALAHKYH.

In some embodiments of the foregoing aspects, the SCD includes complement deposition in red blood cells (RBC). For example, in some embodiments, the SCD includes C5b9 deposition in RBC.

In some embodiments of the foregoing aspects, the SCD includes intravascular hemolysis (IVH). In some embodiments, the IVH is characterized by an increase in at least one marker including LDH, bilirubin, free hemoglobin, and free heme.

In some embodiments of any of the foregoing aspects, upon administration of the complement alternative pathway inhibitor to the subject, the subject exhibits a reduction in a SCD, a BT or a sickle cell BT phenotype. For example, in some embodiments, the SCD phenotype includes increased inflammation or cytotoxicity leading to vascular tissue damage; enhanced pain triggered by VOC events; or increases in mortality or morbidity of SCD patients.

In some embodiments of any of the foregoing aspects, the composition is administered intravenously.

In another aspect, the disclosure features a method for improving viability or reducing death of cells under hypoxic conditions including contacting the cells with an effective amount of a composition including a complement alternative pathway inhibitor.

In some embodiments of the foregoing aspect, the cells are contacted in vivo.

In some embodiments of the foregoing aspect, the cells are sickle cells

In some embodiments of any of the foregoing aspects, SCD is characterized by a feature selected from: (a) increased deposition of complement C3 and/or C5b9 in affected cells (e.g., RBCs), especially under a trigger (e.g., hypoxia); (b) increased neovascular hemolysis, especially under a trigger (e.g., hypoxia), wherein increased hemolysis is characterized by increases in plasma LDH activity/levels, free heme and/or free hemoglobin levels, and/or total bilirubin levels; or (c) increased severity of VOC, especially under a trigger (e.g., hypoxia).

In some embodiments of any of the foregoing aspects, treatment with a complement inhibitor results in an outcome selected from: (a) inhibition or reversal of complement fragment deposition of C3 and C5b9 in RBCs of the subject with SCD, e.g., under hypoxic conditions; (b) attenuation or reversal in the level of intravascular hemolysis under hypoxic conditions (as measured increases in plasma LDH activity/levels, free heme and/or free hemoglobin levels, and/or total bilirubin levels); or (c) reduction or reversal in vaso-occlusion in the vessels of vital organs such as lung, kidney, liver and spleen of the subject with SCD. For example, in some embodiments, treatment with a complement inhibitor results in an improvement in an at least one outcome from (a)-(c) compared to treatment of the subject with hydroxyurea.

In another aspect, the disclosure features a composition including a complement alternative pathway inhibitor for use in treating SCD or a symptom related thereto in a subject, particularly for improving viability of blood cells harboring one or mutations that renders them susceptible to hypoxia or low oxygen tension, e.g., mutation of normal hemoglobin A (α2ß2) to hemoglobin S (α2ß 6 Val2) or mutation in the β-globulin gene of RBC.

In another aspect, the disclosure features a composition including a complement alternative pathway inhibitor for use in improving viability or reducing death of cells under hypoxic conditions.

In some embodiments of the foregoing aspect, the complement alternative pathway inhibitor is a properdin inhibitor.

In some embodiments of the foregoing aspect, the properdin inhibitor is selected from the group including an anti-properdin antibody or a bi-specific antibody including at least one moiety that binds to properdin.

In some embodiments of any of the foregoing aspects, the complement alternative pathway inhibitor is a nucleic acid molecule selected from the group consisting of small interfering RNA, short hairpin RNA, micro RNA and antisense oligonucleotide. In some embodiments, the nucleic acid molecule is complementary to a portion of an endogenous nucleic acid sequence encoding complement C3.

The present disclosure is based, at least in part, on the surprising discovery that complement inhibitors (e.g., a properdin inhibitor e.g., an anti-properdin antibody, a nucleic acid molecule, a peptide, a small molecule, or an aptamer) provide a surprising ability to attenuate the pathogenesis associated with SCD, BT, or sickle cell BT. Comparative assessment of anti-properdin antibody therapy compared to a standard treatment regimen comprising hydroxyurea (HU) showed that the anti-properdin antibody was superior to HU with respect to attenuation of C3 deposition and the concomitant C5b9 deposition in sickle cell mice under hypoxic conditions. Using the compositions and methods described herein, a complement protein (e.g., properdin) can be efficaciously inhibited for the treatment of SCD, BT, or sickle cell BT.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a more complete understanding of the principles disclosed herein, and the advantages thereof, reference is now made to the following descriptions taken in conjunction with the accompanying drawings.

FIG. 1 shows complement alternative pathway (CAP) on sickle red blood cells (RBC) contribute to sickle cell pathology. RBCs are a site of CAP activation that triggers C3 opsonization on the surface as well as complement-mediated RBC hemolysis. Intravascular hemolysis not only causes anemia but also contributes to further amplification of CAP activation by releasing free heme from RBCs. C3 opsonization of sickle RBCs also promotes anemia through extravascular hemolysis. Furthermore, C3 opsonization is a key mechanistic basis for VOC, as evidenced by the fact that C3 opsonization can be precipitated by exposure of phosphatidyl serine (PS) on sickle RBCs and contributes to VOC by enhancing its interaction with adhesion molecules such as P-selectin and complement receptor 3 (CR3 or Mac-1) on activated endothelial cells.

FIG. 2 shows an experimental outline for studying the effect of inhibition of complement activation in VOC in an in vivo mouse model of SCD. Townes SS mice are prophylactically treated with either PBS (vehicle) or “14E1” (anti-properdin) four times from ten days before hypoxia treatment and sacrificed after hypoxia treatment followed by one hour resting in normoxic condition. In a vehicle-treated subgroup, animals were not exposed to hypoxic condition and continuously maintained in normoxic condition (baseline). Upon euthanasia, blood samples and critical organs were harvested from animals to measure the level of complement deposition on RBCs, intravascular hemolysis and the severity of VOC.

FIG. 3 shows bar charts showing flow cytometry-based analyses of hypoxia-induced complement fragment deposition on sickle cell RBCs exposed to hypoxic conditions and the effect of anti-properdin monoclonal antibodies on complement deposition. Shown are changes in complement fragment levels, from left to right, under normal, hypoxic (control), hypoxic+hydroxyurea, and hypoxic+anti-properdin (14E1) pretreatment. The right-hand panel shows C3/C3b/iC3b levels and the left-hand panel shows C5b9 levels.

FIG. 4 shows bar charts showing effects of 14E1 monoclonal antibodies against hypoxia-induced intravascular hemolysis in SCD animals. Shown are changes in hemolysis marker levels, from left to right, under normal, hypoxic (control), hypoxic+hydroxyurea, and hypoxic+anti-properdin (14E1) pretreatment. The following hemolysis markers were measured: lactate dehydrogenase (LDH)(top left-hand panel); bilirubin (bottom right-hand panel); free hemoglobin (bottom left-hand panel); and free heme (top right-hand panel).

FIG. 5 shows data on hypoxia-induced vaso-occlusion in the lung and the effect of 14E1 monoclonal antibody treatment. On the left are representative photomicrographs of sickle cell (SS) RBCs in the lung of mice under the various conditions (from top to bottom): normoxic, hypoxic (control), hypoxic+hydroxyurea, and hypoxic+14E1 pretreatment. PE anti-mouse TER-119 and DAPI were used as fluorescent probes. The right panel shows a bar graph quantifying fluorescence density of the images using standard software.

FIG. 6 shows data on hypoxia-induced vaso-occlusion in the kidney and the effect of 14E1 monoclonal antibody treatment. On the left are representative photomicrographs of SS RBCs in the kidney of mice under various conditions (from left to right): normoxic, hypoxic (control), hypoxic+hydroxyurea, and hypoxic+14E1 pretreatment. PE anti-mouse TER-119 and DAPI were used as fluorescent probes. The right panel shows a bar graph quantifying fluorescence density of the images using standard software.

FIG. 7 shows data on hypoxia-induced vaso-occlusion in the liver and the effect of 14E1 monoclonal antibody treatment. On the left are representative photomicrographs of SS RBCs in the liver of mice under various conditions (from left to right): normoxic, hypoxic (control), hypoxic+hydroxyurea, and hypoxic+14E1 pretreatment. PE anti-mouse TER-119 and DAPI were used as fluorescent probes. The right panel shows a bar graph quantifying fluorescence density of the images using standard software.

FIG. 8 shows data on hypoxia-induced vaso-occlusion in the spleen and the effect of 14E1 monoclonal antibody treatment. On the left are representative photomicrographs of SS RBCs in the spleen of mice under various conditions (from left to right): normoxic, hypoxic (control), hypoxic+hydroxyurea, and hypoxic+14E1 pretreatment. PE anti-mouse TER-119 and DAPI were used as fluorescent probes. The right panel shows a bar graph quantifying fluorescence density of the images using standard software.

FIG. 9 shows an experimental outline for studying the effect of inhibition of complement activation in VOC in an in vivo mouse model of SCD. Townes SS mice were divided into five groups and prophylactically treated with PBS (vehicle), or 14E1 monoclonal antibody four times from ten days before heme treatment. Animals were exposed to 50 μmol/Kg of heme for three hours after which the animals were sacrificed. In one of the vehicle-treated group, animals were not exposed to heme and served as a baseline. Upon euthanasia, blood samples and critical organs were harvested from the animals to measure the level of complement deposition on RBCs, intravascular hemolysis and the severity of vaso-occlusions.

FIG. 10 shows bar charts showing effects of anti-properdin antibodies against heme-induced intravascular hemolysis in SCD animals. Shown are changes in hemolysis marker levels, from left to right, under normal (control), heme, heme+anti-properdin antibody pretreatment. The following hemolysis markers were measured: bilirubin (far left); lactate dehydrogenase (LDH) (center); and free hemoglobin (far right). ****P<0.0001; ***P<0.001; **P<0.01; *P<0.05.

FIG. 11 shows bar charts showing effects of anti-properdin antibodies against heme-induced intravascular hemolysis in SCD animals. Shown are changes in complement fragment levels, from left to right, normal, heme, and heme+anti-properdin antibody pretreatment. The left-hand panel shows C3/C3b/iC3b deposition and the right-hand panel shows C5b9 deposition. ***P<0.001; **P<0.01; *P<0.05.

FIG. 12 shows data on heme-induced vaso-occlusion in the lung and the effect of anti-properdin antibody treatment. On the left are representative photomicrographs of sickle cell (SS) RBCs in the lung of mice under the various conditions (from left to right): normal (control), heme, and heme+anti-properdin antibody pretreatment. The right panel shows a bar graph quantifying fluorescence density of the images using standard software. ****P<0.0001; ***P<0.001.

FIG. 13 shows data on heme-induced vaso-occlusion in the liver and the effect of anti-properdin antibody treatment. On the left are representative photomicrographs of sickle cell (SS) RBCs in the lung of mice under the various conditions (from left to right): normal (control), heme, and heme+anti-properdin antibody pretreatment. The right panel shows a bar graph quantifying fluorescence density of the images using standard software. ****P<0.0001; ***P<0.001; *P<0.05.

FIG. 14 shows flow cytometry-based data on heme-induced complement deposition on sickle RBCs and the effect of anti-properdin antibody treatment. On the left are scatterplots showing iC3b deposition under various conditions, including normal, heme, and heme+anti-properdin antibody. On the right is a bar graph quantifying the iC3b deposition. ****P<0.0001.

FIG. 15 shows flow cytometry-based data on heme-induced complement deposition on sickle RBCs and the effect of anti-properdin antibody treatment. On the left are scatterplots showing C5b9 deposition under various conditions, including normal, heme, and heme+anti-properdin antibody pretreatment. On the right is a bar graph quantifying the C5b9 deposition. **P<0.01.

FIG. 16 shows bar charts showing flow cytometry-based analyses of heme-induced complement fragment deposition on endothelial cells exposed to heme and the effect of anti-properdin antibodies on complement deposition. Shown are changes in complement fragment levels, from left to right, normal, heme, and heme+anti-properdin antibody pretreatment. The left-hand panel shows C3/C3b/iC3b deposition and the right-hand panel shows C5b9 deposition. ns=not significant. ****P<0.0001.

### DETAILED DESCRIPTION

The instant disclosure is based, in part, on the finding of the role of the complement protein, factor P (properdin), in the development and/or manifestation of Sickle cell disease (SCD), a life-threatening disease with poor quality of life for patients. Utilizing recognized animal models (e.g., Towne's SCD mouse model, wherein mouse hemoglobin α and β genes are replaced with the corresponding human genes containing sickle cell mutation (β^{S}) with a single amino acid replacement (Glu→Val)), the present application demonstrates, for the first time, a hitherto unrecognized role of a complement alternative pathway (CAP) inhibitor (e.g., an anti-properdin antibody), for the effective amelioration of SCD or symptoms related thereto in vivo.

### Definitions

Before describing the disclosure in detail, it is to be understood that this disclosure is not limited to particular compositions or biological systems, which can, of course, vary. It is also to be understood that the terminology used herein is for the purpose of describing particular embodiments only and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “and/or” includes any and all possible combinations of one or more of the associated listed items, as well as the lack of combinations when interpreted in the alternative (“or”).

It is understood that aspects and embodiments of the disclosure described herein include “comprising,” “consisting,” and “consisting essentially of” aspects and embodiments.

The term “about” means a range of plus or minus 10% of that value, e.g., “about 5” means 4.5 to 5.5, unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5.

The term “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance (e.g., +/−10%).

Where a range of values is provided in this disclosure, it is intended that each intervening value between the upper and lower limit of that range and any other stated or intervening value in that stated range is encompassed within the disclosure. For example, if a range of 1 mM to 8 mM is stated, it is intended that 2 mM, 3 mM, 4 mM, 5 mM, 6 mM, and 7 mM are also explicitly disclosed.

The term “subject” can be any animal, e.g., a mammal. A subject can be, for example, a human, a non-human primate (e.g., monkey, baboon, or chimpanzee), a horse, a cow, a pig, a sheep, a goat, a dog, a cat, a rabbit, a guinea pig, a gerbil, a hamster, a rat, or a mouse. Included are, e.g., transgenic animals or genetically altered (e.g., knock-out or knock-in) animals.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment, e.g., a particular therapeutic or prophylactic or diagnostic agent to treat a complement-mediated disease or disorder.

As used herein, the terms “treat” or “treating” refer to providing an intervention, e.g., providing any type of medical or surgical management of a subject. The treatment can be provided to reverse, alleviate, inhibit the progression of, prevent or reduce the likelihood of a disorder or condition, or to reverse, alleviate, inhibit or prevent the progression of, prevent or reduce the likelihood of one or more symptoms or manifestations (e.g., pathophysiology) of a disorder or condition. “Prevent” refers to causing a disorder or condition, or symptom or manifestation of such not to occur for at least a period of time in at least some individuals. Treating can include administering a complement inhibitor (e.g., a properdin inhibitor) to the subject following the development of one or more symptoms or manifestations indicative of a complement-mediated condition, e.g., to reverse, alleviate, reduce the severity of, and/or inhibit or prevent the progression of the condition and/or to reverse, alleviate, reduce the severity of, and/or inhibit or one or more symptoms or manifestations of the condition. According to the methods described herein, a complement inhibitor (e.g., a properdin inhibitor) can be administered to a subject who has developed a complement-mediated disease or is at increased risk of developing such a disorder relative to a member of the general population. Such an inhibitor (e.g., a properdin inhibitor) can be administered prophylactically, i.e., before development of any symptom or manifestation of the condition. Typically, in this case, the subject will be at risk of developing the condition, for example, when exposed to a complement-activating condition, e.g., hypoxia.

The term “symptom” refers to an indication of disease, illness, injury, or that something is not right in the body. Symptoms are felt or noticed by the individual experiencing the symptom, but may not easily be noticed by others, e.g., non-health-care professionals. The term “sign” also refers an indication that something is not right in the body, which can be seen by a doctor, nurse, or other health care professional.

The terms “administration” or “administering” when used in conjunction with an agent, e.g., drug, mean to deliver the agent directly into or onto a cell or target tissue or to provide the agent to a patient whereby it impacts the tissue to which it is targeted.

The term “contact” refers to bringing an agent (e.g., anti-properdin antibody) and the target (e.g., factor P) in sufficiently close proximity to each other for one to exert a biological effect on the other (e.g., inhibition of the target). In some embodiments, the term contact means binding of the agent to the target.

The terms “inhibitor” or “antagonist” as used herein refer to a substance, such as an antibody, nucleic acid, aptamer, and small molecule, that suppress the expression, activity, and/or level of another substance (e.g., a complement component, such as properdin). Functional or physiological antagonism occurs when two substances produce opposite effects on the same physiological function. Chemical antagonism or inactivation is a reaction between two substances to neutralize their effects, e.g., binding of an antibody to an antigen, which prevents the antigen from acting on its target. Dispositional antagonism is the alteration of the disposition of a substance (its absorption, biotransformation, distribution, or excretion) so that less of the agent reaches the target or its persistence there is reduced. The term “inhibit” or “reduce” or grammatical variations thereof refers to a decrease or diminishment in the specified level or activity of the target, e.g., little or essentially no detectible level or activity of the target (at most, an insignificant amount). Examples of inhibitors of this type are antibodies, interfering RNA molecules, such as siRNA, miRNA, and shRNA. In addition to encompassing substances that inhibit the expression of a complement protein (e.g., properdin), additional examples of properdin inhibitors include substances, such as small molecules, that attenuate the transcription of an endogenous gene encoding a complement protein (e.g., properdin). In some embodiments, the inhibitor is not a complement C5 inhibitor.

As used herein, the term “disrupt,” with respect to a gene, refers to preventing the formation of a functional gene product. A gene product is functional if it fulfills its normal (wild-type) functions. Disruption of the gene prevents expression of a functional factor encoded by the gene and may contain an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. The disruption of endogenous properdin can be accomplished e.g., by using anti-properdin antibodies, nucleic acid molecules, siRNA, shRNA, miRNA, antisense oligonucleotide, aptamers, and gene editing techniques.

As used herein, the term “endogenous” describes a molecule (e.g., a metabolite, polypeptide, nucleic acid, or cofactor) that is found naturally in a particular organism (e.g., a human) or in a particular location within an organism (e.g., an organ, a tissue, or a cell, such as a human cell).

As used herein, the term “antibody” means an antibody, or a functional portion or fragment thereof, with a high binding affinity for an antigen, e.g., complement proteins. The term is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rlgG) fragments, single chain antibody fragments, including single chain variable fragments (scFv), and single domain antibodies (e.g., sdAb, sdFv, nanobody) fragments. The term encompasses natural, genetically engineered and/or otherwise modified antibodies of any class or subclass, including IgG and sub-classes thereof, IgM, IgE, IgA, and IgD.

The term “monoclonal antibody,” as used herein, refers to an antibody that displays a single binding specificity and affinity for a particular epitope. Accordingly, the term “human monoclonal antibody,” or “HuMab,” refers to an antibody that displays a single binding specificity and that has variable and constant regions derived from human germline immunoglobulin sequences.

The term “single domain antibody”, also known as domain antibody, VHH, VNAR or sdAb, is a kind of antibody consisting of a single monomeric variable antibody domain and lacking the light chain and CH domain of the heavy chain in conventional Fab region. sdAbs can be generated from, e.g., VHH domains of camelid (e.g., dromedaries, camels, llamas, and alpacas) heavy-chain antibody and VNAR domains of cartilaginous fish (e.g., shark) heavy-chain antibody (known as immunoglobulin new antigen receptor (IgNAR)). Alternately, sdAbs may be generated by splitting dimeric variable domains from normal IgG of humans or mice into monomers by camelizing a few critical residues.

The term “antigen” refers to any molecule, e.g., protein or a fragment thereof, that can specifically bind to an antibody or its antigen-binding fragment.

“Antibody fragments” include a portion of an intact antibody, e.g., the antigen binding or variable region of the intact antibody. Examples of antibody fragments include Fab, Fab′, F(ab′)2, and Fv fragments; diabodies; linear antibodies (Zapata et al. Protein Eng. 8(10):1057-1062 (1995)); single-chain antibody molecules; and multispecific antibodies formed from antibody fragments.

The term “antigen fragment” refers to a part of the antigen that can be recognized by the antigen-specific antibody.

The term “antigen-binding fragment” refers to a part of an antibody molecule that comprises amino acids responsible for the specific binding between antibody and antigen. Antigen-binding fragments typically contain variable heavy chain (VH) complementarity-determining regions (CDR) 1-3 (VHCDR1-3), optionally together with variable light chain (VL) CDRs 1-3 (VLCDR1-3). For certain antigens, the antigen-binding domain or antigen-binding fragment may only bind to a part of the antigen.

The part of the antigen that is specifically recognized and bound by the antibody is referred to as the “epitope” or “antigenic determinant.” Antigen-binding domains and antigen-binding fragments include Fab (fragment antigen-binding); a F(ab′)2 fragment, a bivalent fragment having two Fab fragments linked by a disulfide bridge at the hinge region; Fv fragment; a single chain Fv fragment (scFv) (see, e.g., Bird et al. Science 242:423-426, 1988; and Huston et al. Proc. Natl. Acad. Sci. USA 85:5879-5883, 1988); a Fd fragment having the two VH and CH1 domains; dAb (Ward et al., Nature 341:544-546, 1989). The Fab fragment has VH-CH1 and VL-CL domains covalently linked by a disulfide bond between the constant regions. The Fv fragment is smaller and has VH and VL domains non-covalently linked. To overcome the tendency of non-covalently linked domains to dissociate, a scFv can be constructed. The scFv contains a flexible polypeptide that links (1) the C-terminus of VH to the N-terminus of VL, or (2) the C-terminus of VL to the N-terminus of VH. A 15-mer (Gly4Ser)3 peptide may be used as a linker, but other linkers are known in the art. In the case of camelid antibodies, which do not contain a light chain, the antigen-binding fragment contains the CDRs of the VHH. Antigen-binding fragments can be obtained using conventional techniques, and the fragments are screened for utility in the same manner as are intact antibodies. Antigen-binding fragments can be produced by recombinant DNA techniques, or by enzymatic or chemical cleavage of intact immunoglobulins.

The term “hypervariable region” or “HVR,” as used herein, refers to each of the regions of an antibody variable domain that are hypervariable in sequence and/or form structurally defined loops (“hypervariable loops”). Generally, native four-chain antibodies comprise six HVRs; three in the HCVR (H1, H2, H3), and three in the LCVR (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5^{th} Ed. Public Health Service, National Institutes of Health, Bethesda, MD (1991).) With the exception of CDR1 in HCVR, CDRs generally comprise the amino acid residues that form the hypervariable loops.

As used herein, the term “interfering RNA” refers to a RNA, such as a siRNA, miRNA, or shRNA that suppresses the expression of a target RNA transcript, for example, by way of (i) annealing to the target RNA transcript, thereby forming a nucleic acid duplex; and (ii) promoting the nuclease-mediated degradation of the RNA transcript and/or (iii) slowing, inhibiting, or preventing the translation of the RNA transcript, such as by sterically precluding the formation of a functional ribosome-RNA transcript complex or otherwise attenuating formation of a functional protein product from the target RNA transcript. Interfering RNAs as described herein may be provided to a patient, such as a human patient having SCD or a related disorder described herein, in the form of, for example, a single- or double-stranded oligonucleotide, or in the form of a vector (e.g., a viral vector) containing a transgene encoding the interfering RNA. Exemplary interfering RNA platforms are described, for example, in Lam et al., Mol. Ther. Nucleic Acids 4:e252 (2015); Rao et al., Adv. Drug Deliv. Rev. 61:746-769 (2009); and Borel et al., Mol. 22:692-701 (2014), the disclosures of each of which are incorporated herein by reference in their entirety.

The term “small molecule” refers to an organic molecule having a molecular weight less than about 2500 amu, less than about 2000 amu, less than about 1500 amu, less than about 1000 amu, or less than about 750 amu. In some embodiments a small molecule contains one or more heteroatoms.

The term “aptamer” used herein refers to an oligonucleotide (generally, RNA molecule) linked to a specific target. “Aptamer” can refer to an oligonucleotide aptamer (for example, RNA aptamer). The term “aptamer” as used herein refers to DNA or RNA molecules that have been selected from random pools based on their ability to bind other molecules. Aptamers have been selected that bind nucleic acid, proteins, small organic compounds, and even entire organisms. A database of aptamers is maintained at world-wide-web at aptamer(dot)icmb(dot)utexas(dot)edu/.

As used herein, the term “human properdin” refers to a 469 amino acid soluble glycoprotein found in plasma that has seven thrombospondin type I repeats (TSR) with the N-terminal domain, TSR0, being a truncated domain. Human properdin, a 53 kDa protein, includes a signal peptide (amino acids 1-28), and six, non-identical TSR repeats about 60 amino acids each, as follows: amino acids 80-134 (TSR1), amino acids 139-191 (TSR2), amino acids 196-255 (TSR3), amino acids 260-313 (TSR4), amino acids 318-377 (TSR5), and amino acids 382-462 (TSR6). Properdin is formed by oligomerization of a rod-like monomer into cyclic dimers, trimers, and tetramers. The amino acid sequence of human properdin is found in the GenBank database under the following accession numbers: for human properdin, see, e.g., GenBank Accession Nos. AAA36489, NP-002612, AAH15756, AAP43692, S29126 and CAA40914. Properdin is a positive regulator of the alternative complement activation cascade. Known binding ligands for properdin include C3b, C3bB and C3bBb (Blatt, A. et al., Immunol. Rev., 274:172-90, 2016).

As used herein, the term “mouse properdin” refers to a 457 amino acid soluble glycoprotein found in plasma that has seven TSRs with the N-terminal domain, TSR0, being truncated. Mouse properdin, a 50 kDa protein, includes a signal peptide (amino acids 1-24), and six, non-identical TSRs of about 60 amino acids each, as follows: amino acids 73-130 (TSR1), amino acids 132-187 (TSR2), amino acids 189-251 (TSR3), amino acids 253-309 (TSR4), amino acids 311-372 (TSR5), and amino acids 374-457 (TSR6). Mouse properdin is formed by oligomerization of a rod-like monomer into cyclic dimers, trimers, and tetramers. The amino acid sequence of mouse properdin is found, for example, in the GenBank database (GenBank Accession Nos. P11680 and S05478).

As used herein, the term “alternative complement pathway” refers to one of three pathways of complement activation (the others being the classical pathway and the lectin pathway). The alternative complement pathway is typically activated by bacteria, parasites, viruses or fungi, although IgA Abs and certain IgL chains have also been reported to activate this pathway.

As used herein, the term “alternative complement pathway dysregulation” refers to any aberration in the ability of the alternative complement pathway to provide host defense against pathogens and clear immune complexes and damaged cells and for immunoregulation. Alternative complement pathway dysregulation can occur both in fluid phase as well as at cell surface and can lead to excessive complement activation or insufficient regulation, both causing tissue injury.

As used herein, the term “a disease mediated by alternative complement pathway dysregulation” refers to an interruption, cessation or disorder of body functions, systems or organs caused by alternative complement pathway dysregulation. Such diseases would benefit from treatment with a composition or formulation described herein. In some embodiments, the disease is caused by any aberration in the ability of the alternative complement pathway to provide host defense against pathogens and clear immune complexes and damaged cells, and for immunoregulation. Also encompassed herein are diseases, directly or indirectly, mediated by dysregulation of one or more components of the alternative complement pathway, or a product generated by the alternative complement pathway.

As used herein, the term “alternative complement pathway-dependent membrane attack complex assembly” refers to a terminal complex formed as a result of alternative complement pathway activation and includes complement components C5, C6, C7, C8, and C9. Assembly of the membrane attack complex (MAC) leads to cell lysis.

As used herein, the term “alternative complement pathway dependent hemolysis” refers to the lysis of red blood cells mediated by increased alternative complement pathway-dependent MAC assembly and/or deposition on red blood cells.

The terms “sample” or “biological sample” are meant to mean any entity obtained from a subject (e.g., composition containing cells, blood, plasma, serum or other blood fractions, lymph, urine, cerebrospinal fluid, ascites, saliva, breast milk, tears, vaginal discharge, amniotic fluid, lavage, semen, glandular secretions, exudate, contents of cysts and feces).

An “effective amount” of an active agent, such as a complement inhibitor (e.g., a properdin inhibitor), refers to the amount of the active agent sufficient to elicit a desired biological response (or, equivalently, to inhibit an undesired biological response). The absolute amount of a particular agent that is effective may vary depending on such factors as the desired biological endpoint, the agent to be delivered, the target tissue, etc. An “effective amount” may be administered in a single dose or administration of multiple doses. An effective amount of the therapeutic agent, for example, may be an amount sufficient to relieve at least one symptom of a disorder. An effective amount may be an amount sufficient to slow the progression of a chronic and progressive disorder, e.g., to increase the time before one or more symptoms or signs of the disorder manifests itself or to increase the time before the individual suffering from the disorder reaches a certain level of impairment. An effective amount may be an amount sufficient to allow faster or greater recovery from a disease than would occur in the absence of the agent. For purposes of this disclosure, an effective amount of drug, compound, or pharmaceutical composition is an amount sufficient to accomplish prophylactic or therapeutic treatment either directly or indirectly. As is understood in the clinical context, an effective amount of a complement inhibitor (e.g., a properdin inhibitor) or pharmaceutical composition thereof may or may not be achieved in conjunction with another drug, compound, or pharmaceutical composition. Thus, an “effective amount” may be considered in the context of administering one or more therapeutic agents, and a single complement inhibitor (e.g., a properdin inhibitor) may be considered to be given in an effective amount if, in conjunction with one or more other agents, a desirable result may be or is achieved.

As used herein, “activity” refers to form(s) of a polypeptide that retain a biological activity of the native or naturally-occurring polypeptide, wherein “biological” activity refers to a biological function (e.g., enzymatic function) caused by a native or naturally-occurring polypeptide.

By “pharmaceutically acceptable” carrier is meant a carrier comprised of a material that is not biologically or otherwise undesirable. The term “carrier” is used generically herein to refer to any components present in the pharmaceutical formulations other than the active agent or agents, and thus includes diluents, binders, lubricants, disintegrants, fillers, coloring agents, wetting or emulsifying agents, pH buffering agents, preservatives, and the like. Similarly, a “pharmaceutically acceptable” salt or a variant (e.g., ester) of a molecule as provided herein is one that is not biologically or otherwise undesirable.

As used herein, the term “salt” refers to the relatively non-toxic, inorganic and organic acid addition salts of compounds of the presently disclosed subject matter. These salts can be prepared in situ during the final isolation and purification of the compounds or by separately reacting the purified compound in its free base form with a suitable organic or inorganic acid and isolating the salt thus formed. Pharmaceutically acceptable base addition salts may be formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenyl acetate, citrate, lactate, maleate, tartrate, methanesulfonate, and the like.

The term “pharmaceutically acceptable salt” or a variant thereof, as used herein, refers to those salts that are, within the scope of sound medical judgment, suitable for use in contact with subjects (e.g., human subjects) without undue toxicity, irritation, allergic response, and the like, commensurate with a reasonable benefit/risk ratio, and effective for their intended use, as well as the zwitterionic forms, where possible, of the compounds of the present disclosure. Thus, pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like.

As used herein, the term “diagnosis” refers to methods by which a determination can be made as to whether a subject is likely to be suffering from a given disease or condition, including but not limited to SCD and related diseases and disorders. The skilled artisan often makes a diagnosis on the basis of one or more diagnostic indicators, e.g., a marker, the presence, absence, amount, or change in amount of which is indicative of the presence, severity, or absence of the disease or condition. Other diagnostic indicators can include patient history; physical symptoms, e.g., unexplained changes in vitals, or phenotypic, genotypic or environmental or heredity factors. A skilled artisan will understand that the term “diagnosis” refers to an increased probability that certain course or outcome will occur; that is, that a course or outcome is more likely to occur in a patient exhibiting a given characteristic, e.g., the presence or level of a diagnostic indicator, when compared to individuals not exhibiting the characteristic. Diagnostic methods of the disclosure can be used independently, or in combination with other diagnosing methods, to determine whether a course or outcome is more likely to occur in a patient exhibiting a given trait.

The term “cell” refers to basic building blocks of tissue, such as cells from a human, monkey, mouse, rat, rabbit, hamster, goat, pig, dog, cat, ferret, cow, sheep, horse or the like. The cells may be diploid or haploid (i.e., sex cells). The cells may also be polyploid, aneuploid, or anucleate. The cell may be from a particular tissue or organ, such as blood, heart, lung, kidney, liver, bone marrow, pancreas, skin, bone, vein, artery, cornea, blood, small intestine, large intestine, brain, spinal cord, smooth muscle, skeletal muscle, ovary, testis, uterus, umbilical cord or the like. The cell may also be a platelet, myelocyte, erythrocyte, lymphocyte, adipocyte, fibroblast, epithelial cell, endothelial cell, smooth muscle cell, heart muscle, skeletal muscle cell, endocrine cell, glial cell, neuron, secretory cell, barrier function cell, contractile cell, absorptive cell, mucosal cell, limbus cell, stem cell (totipotent, pluripotent or multipotent), unfertilized or fertilized oocyte, sperm or the like. Included are normal cells and transformed cells.

The terms “sickle cell disease” or “SCD” have their general meaning in the art and refers to a hereditary blood disorder in which red blood cells assume an abnormal, rigid, sickle shape. Sickling of erythrocytes decreases the cells' flexibility and results in a risk of various life-threatening complications. The term includes sickle cell anemia, hemoglobin SC disease and sickle cell beta-thalassemia.

By “beta thalassemia” or “p thalassemia” as used herein is meant a hereditary blood disorder that is due to reduced or absent synthesis of the beta chains of hemoglobin. It is the result of one or more mutations in or near the β globin gene. The terms “vaso-occlusion” or “VOC” have their general meaning in the art, e.g., relating to a common complication of SCD that leads to the occlusion of capillaries and the restriction of blood flow to an organ, resulting in ischemia, with vascular dysfunction, tissue necrosis, and/or organ damage. VOC are usually a constituent of vaso-occlusive crises, but they may also be more limited, clinically silent, and not cause hospitalization for vaso-occlusive crisis. As used herein, the term “vaso-occlusive crisis” refers to a painful complication of SCD that leads to hospitalization, in association with occlusion of capillaries and restriction of blood flow to an organ resulting in ischemia, severe pain, necrosis, and organ damage.

The term “acute chest syndrome” is a condition typically characterized by fever, chest pain, and appearance of a new infiltrate on chest radiograph. The term “chronic lung disease” in the context of SCD typically manifests as radiographic interstitial abnormalities, impaired pulmonary function, and, in its most severe form, by the evidence of pulmonary hypertension.

The term “hemolytic anemia” as used herein refers to any condition in which the number of erythrocytes (RBC) per mm or the amount of hemoglobin in 100 mL of blood is less than normal, e.g., resulting from the destruction of erythrocytes. The term “thrombocytopenia” as used herein refers to a condition in which the number of platelets circulating in the blood is below the normal range of platelets.

The term “complement deposition” refers to an activity or event that leads to the complements, e.g., C5b9 or C3b, to deposit on a target cells (e.g., RBC) by such a manner as to trigger a series of cascades (complement activation pathways) containing complement-related protein groups in blood. In addition, protein fragments generated by the activation of a complement can induce the migration, phagocytosis and activation of immune cells. Related downstream events include, e.g., (a) hemolysis of target cells, leading to heme release and/or anemia in blood cells; or (b) C3 opsonization, which may lead to phagocytosis and extra-vascular hemolysis (EVH); adhesion of opsonized cells to activated endothelium; and/or activation of neutrophils and platelets.

The term “trigger” in the context of SCD include any events or phenomena that initiate, propagate, or exacerbate disease symptom or pathology such as vaso-occlusive crises. Representative examples include, e.g., acidosis, hypoxia and dehydration, all of which potentiate intracellular polymerization of SS hemoglobin (J. H. Jandl, Blood: Textbook of Hematology, 2^{nd} Ed., Little, Brown and Company, Boston, 1996, pages 544-545).

By “determining the level of a nucleic acid” is meant the detection of a nucleic acid (e.g., mRNA) by methods known in the art either directly or indirectly. Methods to measure mRNA level generally include, but are not limited to, northern blotting, nuclease protection assays (NPA), in situ hybridization (ISH), RT-PCR, and RNA sequencing (RNA-Seq).

By “determining the level of a protein” is meant the detection of a protein by methods known in the art either directly or indirectly. Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners.

The term “hemolytic disease” refers to any disorder or disease in which cellular lysis, cellular damage and inflammation play a role in the pathology of the disease. Hemolytic disease is also an inflammatory disorder or disease wherein alternate pathway (AP) activation causes cellular lysis, cellular damage, and inflammation. Hemolytic diseases include diseases characterized by pathologic lysis of erythrocytes and/or platelets. Anucleated cells such as erythrocytes and platelets are subject to full lysis. Lysis of erythrocytes releases many markers, e.g., heme, hemoglobin, LDH, bilirubin, some of which may have pathological outcome for blood and organs. Nucleated cells such as neutrophils, monocytes, T lymphocytes can be attacked by the MAC but do not undergo full lysis. The term “intravascular hemolysis” refers to the lysis of anucleated and nucleated cells that is caused by AP activation and the associated production and deposition of C5b-9 on cell surfaces. The term “extravascular hemolysis” refers to lysis of cells due to C3b deposition and removal via complement receptors. C3b is produced via the activation of the classical and the alternative pathway. This disclosure relates to C3b produced via the alternative complement pathway.

The term “intravenous” generally means “within a vein” and refers to accessing a subject's target cells or tissue via the vasculature system. In intravenous (IV) therapy, liquid substances are administered directly into a vein. Compared with other routes of administration, the intravenous route is probably the fastest way to deliver agents throughout a body. Some medications, blood transfusions, and parenteral (e.g., non-alimentary) nutrients are administered intravenously using standard delivery systems.

The term “hypoxic” refers to conditions where the oxygen level is lower than normal, such as, less than 21%, 15%, 12%, 9%, 6%, 3%, or 2% of normal oxygen level. In contrast, “normoxic” refers to conditions where the oxygen level is substantially close to normal, e.g., within +/−10% of normal levels.

As used herein, the term “detecting,” refers to the process of determining a value or set of values associated with a sample by measurement of one or more parameters in a sample and may further comprise comparing a test sample against reference sample. In accordance with the present disclosure, the detection of complement markers includes identification, assaying, measuring and/or quantifying one or more markers.

The term “likelihood,” as used herein, generally refers to a probability, a relative probability, a presence or an absence, or a degree.

As used herein, the term “marker” refers to a characteristic that can be objectively measured as an indicator of normal biological processes, pathogenic processes, or a pharmacological response to a therapeutic intervention, e.g., treatment with a complement inhibitor. Representative types of markers include, for example, molecular changes in the structure (e.g., sequence or length) or number of the marker, comprising, e.g., changes in level, concentration, activity, or properties of the marker.

The term “control,” as used herein, refers to a reference for a test sample, such as control healthy subjects or untreated subjects, and the like. A “reference sample,” as used herein, refers to a sample of tissue or cells that may or may not have a disease that are used for comparisons. Thus a “reference” sample thereby provides a basis to which another sample, for example, blood from SCD patient, can be compared. In contrast, a “test sample” refers to a sample compared to a reference sample. The reference sample need not be disease free, such as when reference and test samples are obtained from the same patient separated by time.

The term “level” can refer to binary (e.g., absent/present), qualitative (e.g., absent/low/medium/high), or quantitative information (e.g., a value proportional to number, frequency, or concentration) indicating the presence of a particular molecular species. By a “decreased level” or an “increased level” of a protein or nucleic acid (e.g., mRNA) is meant a decrease or increase in protein or nucleic acid (e.g., mRNA) level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, ng/mL) or percentage relative to total protein or nucleic acid (e.g., mRNA) in a sample.

As used herein, the term “at risk” for a disease or disorder refers to a subject (e.g., a human) that is predisposed to experiencing a particular disease. This predisposition may be genetic (e.g., or due to other factors (e.g., environmental conditions, hypertension, activity level, metabolic syndrome, etc.). Thus, it is not intended that the present disclosure be limited to any particular risk, nor is it intended that the present disclosure be limited to any particular type of disorder or dysfunction related to complement (e.g., sickle cell disease).

As used herein, “in conjunction with” refers to administration of one treatment modality in addition to another treatment modality. As such, “in conjunction with” refers to administration of one treatment modality before, during, or after administration of the other treatment modality to the individual.

The term “pharmaceutical composition” refers to a preparation that is in such form as to permit the biological activity of an active ingredient contained therein to be effective, and that contains no additional components that are unacceptably toxic to a subject to which the formulation would be administered.

As used herein, the terms “specific binding,” “selective binding,” “selectively binds,” and “specifically binds,” refer to antibody binding to an epitope on a predetermined antigen. Typically, the antibody binds with an affinity (KD) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 3000 instrument, which can be performed, for example, using recombinant CDH11 as the analyte and the antibody as the ligand. In some embodiments, binding by the antibody to the predetermined antigen is with an affinity that is at least two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody that binds specifically to an antigen.”

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, the terms “transduction” and “transduce” refer to a method of introducing a viral vector construct or a part thereof into a cell and subsequent expression of a transgene encoded by the vector construct or part thereof in the cell.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, diethylaminoethyl (DEAE)-dextran transfection, NUCLEOFECTION™, squeeze-poration, sonoporation, optical transfection, MAGNETOFECTION™, impalefection, and the like.

As used herein, the term “vector” is meant to include, but is not limited to, a nucleic acid molecule that expresses a gene or coding sequence of interest, e.g., a coding sequence that encodes an antibody. Accordingly, one type of vector is a viral vector, wherein additional DNA segments (e.g., transgenes, e.g., transgenes encoding the properdin inhibitor of the disclosure) may be ligated into the viral genome, and the viral vector may then be administered (e.g., by electroporation, e.g., electroporation into muscle tissue) to the subject to allow for transgene expression in a manner analogous to gene therapy.

Another type of vector is a “plasmid,” which refers to a circular double stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids.

### Complement System in Pathology

The complement system acts in conjunction with other immunological systems of the body to defend against intrusion of cellular and viral pathogens. While a properly functioning complement system provides a robust defense against infecting microbes, inappropriate regulation or activation of the complement pathways has been implicated in the pathogenesis of a variety of disorders.

For example, the first report that complement activation may be involved in SCD was published in 1967 (Francis and Womack. Am. J. Med. Technol. 1967; 33(2):77-86). Since then, studies have reported increased levels of complement-derived fragments in the blood of SCD patients, demonstrating that complement is activated in SCD and suggesting that complement may play an important role in the pathophysiology of the disease.

SCD pathology is known to arise from a missense mutation within the β-globin gene, leading to the substitution of valine for glutamic acid on the outer surface of the globin molecule. This amino acid substitution renders the sickle cell hemoglobin (HbS) less soluble and prone to polymerization upon deoxygenation. Erythrocytes (e.g., red blood cells; RBC) carrying polymerized HbS are thus less deformable and may obstruct microvessels. This vascular occlusion, producing tissue ischemic and infarction, represents a major cause of morbidity and mortality among SCD patients. Clinical manifestations of SCD extend far beyond the homozygous globin mutation. Seminal findings were the discovery that sickle (SS) RBCs, unlike normal RBCs, can adhere to stimulated endothelium in vitro and that SS-RBCs' adhesion correlates with the clinical severity of SCD. Subsequent studies have recognized the importance of plasma factors, such as complement proteins, in SS-RBC adhesion to the endothelium. In model systems of SCD, it has been shown that one of the complement proteins, C5a, is activated following the induction of hypoxia/re-oxygenation (e.g., see Vercellotti et al., Am. J. Hematol, 94:3 (2019), 327-338), further suggesting that complement proteins may be directly involved in the pathogenesis of this disorder. Importantly, however, the direct causal role of the complement system in the pathogenesis of SCD or a model thereof has yet to be demonstrated.

Mutations in the β-globin gene also cause other pathologies, including, for example, beta thalassemia (BT). Whereas BT major is caused by both alleles of the beta-globin gene containing a mutation that leads to complete absence of beta globin production, BT intermedia is due to reduced production of beta globin chains and/or production of mutant beta globin chains. BT is a disease that causes chronic anemia (e.g., a shortage of RBCs), which may suggest that complement proteins play an additional role in the pathogenesis of the genetically related disorder BT.

The present disclosure is based, at least in part, on the surprising discovery that complement inhibitors (e.g., a properdin inhibitor e.g., an anti-properdin antibody, a nucleic acid molecule, a peptide, a small molecule, or an aptamer) provide a surprising ability to attenuate the pathogenesis associated with SCD, BT, or sickle cell BT. As described herein, this disclosure is based, at least in part, on the discovery that pre-treatment with a complement inhibitor (e.g., a properdin inhibitor) effectively attenuated SCD-associated pathogenesis, including hypoxia-induced C5b9 deposition, intravascular hemolysis (IVH), and the extent of clogging the vessels in vital organs such as the lungs and liver. These properties are particularly beneficial in view of the prevalence of the pathophysiology of SCD, which includes anemia, oxidative stress, hemolysis, inflammation and vaso-occlusion. Using the compositions and methods described herein, a complement protein (e.g., properdin) can be efficaciously inhibited for the treatment of SCD.

### Complement Proteins

There are at least 25 complement proteins, which are a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory and lytic functions.

The complement cascade can progress via the classical pathway (CP), the lectin pathway, or the alternative pathway (AP). The CP is typically initiated by antibody recognition of, and binding to, an antigenic site on a target cell. The lectin pathway is typically initiated with binding of mannose-binding lectin (MBL) to high mannose substrates. The AP can be antibody independent and initiated by certain molecules on pathogen surfaces. These pathways converge at the C3 convertase—where complement component C3 is cleaved by an active protease to yield C3a and C3b.

Spontaneous hydrolysis of complement component C3, which is abundant in the plasma fraction of blood, can also lead to AP C3 convertase initiation. This process, known as “tickover,” occurs through the spontaneous cleavage of a thioester bond in C3 to form C3i or C3(H₂O). Tickover is facilitated by the presence of surfaces that support the binding of activated C3 and/or have neutral or positive charge characteristics (e.g., bacterial cell surfaces). Formation of C3(H₂O) allows for the binding of plasma protein Factor B, which in turn allows Factor D to cleave Factor B into Ba and Bb. The Bb fragment remains bound to C3 to form a complex containing C3(H₂O)Bb—the “fluid-phase” or “initiation” C3 convertase. Although only produced in small amounts, the fluid-phase C3 convertase can cleave multiple C3 proteins into C3a and C3b and results in the generation of C3b and its subsequent covalent binding to a surface (e.g., a bacterial surface). Factor B bound to the surface-bound C3b is cleaved by Factor D to form the surface-bound AP C3 convertase complex containing C3b,Bb.

The AP C5 convertase ((C3b)₂,Bb) is formed upon addition of a second C3b monomer to the AP C3 convertase. The role of the second C3b molecule is to bind C5 and present it for cleavage by Bb. The AP C3 and C5 convertases are stabilized by the addition of the trimeric protein properdin. Properdin promotes the association of C3b with Factor B and provides a focal point for the assembly of C3bBb on a surface of cells. Properdin also inhibits the Factor H-mediated cleavage of C3b by Factor I. It binds to preformed alternative pathway C3-convertases; however, properdin binding is not required to form a functioning alternative pathway C3 or C5 convertase.

The CP C3 convertase is formed upon interaction of complement component C1, which is a complex of C1q, C1r and C1s, with an antibody that is bound to a target antigen (e.g., a microbial antigen). The binding of the C1q portion of C1 to the antibody-antigen complex causes a conformational change in C1 that activates C1r. Active C1r then cleaves the C1-associated C1s to generate an active serine protease. Active C1s cleaves complement component C4 into C4b and C4a. Like C3b, the newly generated C4b fragment contains a highly reactive thiol that readily forms amide or ester bonds with suitable molecules on a target surface (e.g., a microbial cell surface). C1s also cleaves complement component C2 into C2b and C2a. The complex formed by C4b and C2a is the CP C3 convertase, which is capable of processing C3 into C3a and C3b. The CP C5 convertase (C4b,C2a,C3b) is formed upon addition of a C3b monomer to the CP C3 convertase.

In addition to its role in C3 and C5 convertases, C3b also functions as an opsonin through its interaction with complement receptors present on the surfaces of antigen-presenting cells such as macrophages and dendritic cells. The opsonic function of C3b is generally considered one of the most important anti-infective functions of the complement system. Patients with genetic lesions that block C3b function are prone to infection by a broad variety of pathogenic organisms, while patients with lesions later in the complement cascade sequence, e.g., patients with lesions that block C5 functions, are found to be more prone only to Neisseria infection, and then only somewhat more prone.

The AP and CP C5 convertases cleave C5 into C5a and C5b. Cleavage of C5 releases C5b, which allows for the formation of the lytic terminal complement complex, C5b-9. C5b combines with C6, C7 and C8 to form the C5b-8 complex at the surface of the target cell. Upon binding of several C9 molecules, the membrane attack complex (MAC, C5b-9, terminal complement complex (“TCC”)) is formed. When sufficient numbers of MACs insert into target cell membranes, the openings they create (MAC pores) mediate rapid osmotic lysis of the target cells.

Cleavage of C5 also releases C5a, which, has been shown to be potent anaphylatoxin and chemotactic factor.

### Complement Pathway Inhibitors

Described herein are compositions that bind to and inhibit a component of the complement pathway and are useful for treating SCD, BT, or sickle cell BT. For example, properdin is a positive regulator of the alternative complement pathway. Described herein are compositions that bind to and inhibit a complement protein (e.g., properdin) and are useful for treating SCD, BT, or sickle cell BT.

A number of approaches are known in the art for determining whether a compound modulates expression or activity of a complement pathway component, for example, to determine whether a compound is a complement inhibitor (e.g., a properdin inhibitor). The complement component activity assay may be cell-based, cell-extract-based (e.g., a microsomal assay), a cell-free assay (e.g., a transcriptional assay), or make use of substantially purified proteins. For example, identification of compounds as complement protein inhibitors can be performed using a complement protein (e.g., properdin) liver microsomal assay, for example, as described by Shanklin et al. Proc. Natl. Acad. Sci. USA 88:2510-2514, 1991 or Miyazaki et al. J. Biol. Chem. 275:30132-30138, 2000. In some instances, liquid-chromatography/mass spectrometry (LC/MS)-based approaches can be used to measure the activity of a complement protein (e.g., properdin activity), for example, as described by Dillon et al. Anal. Chim. Acta. 627(1):99-104, 2008. A high-throughput assay can be used, for example, as described by Soulard et al. Anal. Chim. Acta. 627(1):105-111, 2008. Still further approaches to measure the activity of a complement protein are described in U.S. Pat. No. 7,790,408.

Any suitable method can be used to determine whether a compound binds to a complement pathway component (e.g., properdin), for instance, mass spectrometry, surface plasmon resonance, or immunoassays (e.g., immunoprecipitation or enzyme-linked immunosorbent assay).

Any suitable method can be used to determine whether a compound modulates expression of a complement pathway component (e.g., properdin), for instance, Northern blotting, Western blotting, reverse transcription-polymerase chain reaction (RT-PCR), mass spectrometry, or RNA sequencing.

### Complement Inhibitor Modalities

An alternative complement pathway inhibitor can be selected from a number of different modalities. A complement inhibitor can be an antibody, a nucleic acid molecule (e.g., DNA molecule or RNA molecule, e.g., mRNA or inhibitory RNA molecule (e.g., short interfering RNA (siRNA), micro RNA (miRNA), or short hairpin RNA (shRNA)), or a hybrid DNA-RNA molecule), a peptide, a small molecule (e.g., a properdin small molecule inhibitor), an inhibitor of a signaling cascade, an activator of a signaling cascade, or an epigenetic modifier), or an aptamer. Any of these modalities can be a complement inhibitor directed to target (e.g., to inhibit) function of a complement protein; complement expression; complement binding; or complement signaling. The nucleic acid molecule or small molecule may include a modification. For example, the modification can be a chemical modification, e.g., conjugation to a marker, e.g., fluorescent marker or a radioactive marker. The modification can also include conjugation to an antibody to target the agent to a particular cell or tissue. Additionally, the modification can be a chemical modification, packaging modification (e.g., packaging within a nanoparticle or microparticle), or targeting modification.

### I. Anti-Complement Alternative Pathway Antibodies

Described herein are anti-complement alternative pathway antibodies, antibody derivatives (e.g., engineered antibodies, humaneered antibodies, chimeric antibodies, substituted antibodies, humanized antibodies, etc.) and antibody fragments thereof that inhibit a protein in the complement alternative pathway. The inhibitory antibodies described herein (e.g., neutralizing, blocking, or depleting) can inhibit, for example, a protein in the complement alternative pathway.

For example, described herein are monovalent anti-properdin antibodies, antibody derivatives (e.g., engineered antibodies, humaneered antibodies, chimeric antibodies, substituted antibodies, humanized antibodies, etc.) and antibody fragments thereof that inhibit properdin, a positive regulator of the alternate pathway of complement, and subsequently destabilize the C3- and C5-convertase enzyme complexes. The inhibitory antibodies described herein (e.g., neutralizing, blocking, or depleting) can inhibit, for example, properdin binding to C3b, C3Bb, and C3bBb. For example, an anti-properdin antibody or antigen-binding fragments thereof described herein is an antibody that reduces or blocks the activity and/or function of properdin through binding to properdin. Such polypeptides may have one or more, or all, of the complementary determining regions (CDRs) of inhibitory properdin antibodies described herein (see, e.g., Table 1, below) or one or more of the heavy chains (HC), light chains (LC), heavy chain variable regions (HCVR), or light chain variable regions (LCVR) described herein (see e.g., Table, 2, below). Inhibition of properdin leads to reduced alternative pathway complement activation, indicating a therapeutic benefit for patients afflicted with a disease of alternative pathway dysregulation wherein the alternative pathway is hyper-activated. For example, the anti-properdin antibodies or antigen binding fragments thereof may benefit the treatment of SCD, BT, or sickle cell BT by modulating sickle cell activity.

TABLE 1
Exemplary CDR sequences
of an anti-properdin antibody

| SEQ ID NO: | CDR | SEQUENCE |
|---|---|---|
| 2 | CDR-H1 | GRISSIIHMA |
| 3 | CDR-H2 | RVGTTVYADSVKG |
| 4 | CDR-H3 | LQYEKHGGADY |
| 7 | CDR-H1 | GFSLTTYGVH |
| 8 | CDR-H2 | VIWSGGDTDYNASFIS |
| 9 | CDR-H3 | NKDYYTNYDFTMDY |
| 10 | CDR-L1 | KSSQSVLYSSNQKNFLA |
| 11 | CDR-L2 | WASTRES |
| 12 | CDR-L3 | HQYLSSYT |
| 13 | CDR-H1 | GYTFIDYWIE |
| 14 | CDR-H2 | EIFPGSGTINHNEKFK |
| 15 | CDR-H3 | EGLDY |
| 16 | CDR-L1 | SASSSVSYIY |
| 17 | CDR-L2 | DTSTLAS |
| 18 | CDR-L3 | QQWSRNPFT |
| 19 | CDR-H1 | GFSLTSYGVH |
| 20 | CDR-H2 | VIWSGGSTDYNAAFIS |
| 21 | CDR-H3 | NKDFYSNYDYTMDY |
| 22 | CDR-L1 | KSSQSVLYSSNQKNFLA |
| 23 | CDR-L2 | WASTRES |
| 24 | CDR-L3 | HQYLSSYT |
| 25 | CDR-H1 | GYT*TAYGIN |
| 26 | CDR-H2 | YIYIGNGYTDYNEKFKG |
| 27 | CDR-H3 | SGWDEDYAMDF |
| 28 | CDR-L1 | RASENIYSYLA |
| 29 | CDR-L2 | HAKTLAE |
| 30 | CDR-L3 | QHHYGPPPT |

*can be any naturally occurring amino acid

In some embodiments, the antibodies or antigen-binding fragments thereof comprise a full set of CDRs comprising VHCDR1-3 and VLCDR1-3. For example, in a first aspect the anti-properdin antibody or antigen-binding fragment thereof may comprise VHCDR1-3 sequences comprising SEQ ID Nos: 7, 8 and 9, respectively and VLCDR1-3 sequences comprising SEQ ID Nos: 10, 11 and 12, respectively (FP1). In a second aspect, the anti-properdin antibody or antigen-binding fragment thereof may comprise VHCDR1-3 sequences comprising SEQ ID Nos: 13, 14 and 15, respectively and VLCDR1-3 sequences comprising SEQ ID Nos: 16, 17, and 18 respectively (FP2). In a third aspect, the anti-properdin antibody or antigen-binding fragment thereof may comprise VHCDR1-3 sequences comprising SEQ ID Nos: 19, 20 and 21, respectively and VLCDR1-3 sequences comprising SEQ ID Nos: 22, 23 and 24, respectively (FP3). In a fourth aspect, the anti-properdin antibody or antigen-binding fragment thereof may comprise VHCDR1-3 sequences comprising SEQ ID Nos: 25, 26 and 27, respectively and VLCDR1-3 sequences comprising SEQ ID Nos: 28, 29 and 30, respectively (FP4).

In some embodiments, the disclosure relates to use of monovalent anti-properdin antibodies and antigen-binding fragments thereof. Representative examples are provided in WO2018140956 and US Pub. No. 2019/0352381, the disclosures in which are incorporated by reference herein. In a first aspect, the monovalent antibody or antigen-binding fragment thereof may comprise VHCDR1-3 sequences comprising SEQ ID NOs: 2, 3, and 4, respectively. In one embodiment, the disclosure is directed to an isolated monovalent antibody or antibody fragment thereof, where the antibody or antibody fragment thereof binds human properdin. In a particular embodiment, the antibody or fragment is a camelid antibody. In a particular embodiment, the antibody or fragment is a single-domain antibody. In a particular embodiment, the antibody or fragment inhibits an activity of human properdin.

In some embodiments, the anti-properdin antibody comprises a bispecific antibody, particularly a minibody. Representative types of bispecific anti-properdin minibodies are provided in WO 2018140956, which are incorporated by reference in their entirety. In some embodiments, the bispecific minibody comprises sequences (e.g., CDRs) which bind with specificity to a first antigen (e.g., properdin or an antigenic fragment thereof) and sequences (e.g., CDRs) which bind with specificity to a second antigen, (e.g., albumin or an antigenic fragment thereof). The orientation of the properdin-binding sequences and the albumin-binding sequences may be reversed, i.e., with respect to the amino-to-carboxyl termini of the minibody, the properdin-binding sequence(s) may precede or follow (preferably follow) the albumin-binding sequence(s). In some embodiments, the properdin-binding sequences comprise only the antibody heavy chain CDRs (CDRH1-3) of an anti-properdin antibody, e.g., the sequences of SEQ ID NOs: 2-4, respectively. Preferably, these properdin-binding CDRs are located at the C-terminus of the minibody. In some embodiments, the properdin-binding sequence(s) are linked (e.g., conjugated) to the albumin-binding sequence via a linker, e.g., a linker having the amino acid sequence of SEQ ID NO: 5. In a particular embodiment, the anti-properdin antibody comprises a minibody sequence of SEQ ID NO: 6.

TABLE 2
Exemplary variable region
sequences of an anti-properdin antibody

| SEQ ID NO: | Variable region | SEQUENCE |
|---|---|---|
| 43 | HC | MAVLGLLFCLVTFPSCVLSQV |
| | | QLKQSGPGLVQPSQSLSISCT |
| | | VSGFSLTTYGVHWVRQSPGK |
| | | GLEWLGVIWSGGDTDYNASFI |
| | | SRLRINKDTSKSQVFFKMNSL |
| | | QADDTAYYCARNKDYYTNYDF |
| | | TMDYWGQGTSVTVSS |
| 44 | LC | MESQTQVFLSLLLWVSGTCGN |
| | | IMMTQSPSSLAVSAGEKVTMS |
| | | CKSSQSVLYSSNQKNFLAWYQ |
| | | QKPGQSPKLLIYWASTRESGV |
| | | PDRFTGSGSGTDFILTINSVQV |
| | | EDQAVYYCHQYLSSYTFGGGT |
| | | KLEIK |
| 45 | HC | MDWTWVFLFLLSVTAGVHSQV |
| | | QLLQSGAEVMKPGASVTLSCK |
| | | AIGYTFIDYWIEWIKQRPGHGL |
| | | EWIGEIFPGSGTINHNEKFKDK |
| | | ASFSAHSSSNTAYMQLSRLTS |
| | | EDSAIYYCAREGLDYWGQGTT |
| | | LTVSS |
| 46 | LC | MDFQVQIFSFLLISASVIISRGQI |
| | | VLTQSPAIMSASPGERVTMTC |
| | | SASSSVSYIYWYQQKSGTSPK |
| | | RWIFDTSTLASGVPTRFSGSG |
| | | SGTSYSLTISSMETEDAATYYC |
| | | QQWSRNPFTFGSGTKLEIK |
| 47 | HC | MAVLGLLFCLVTFPSCVLSQV |
| | | QLKQSGPGLVQPSQSLSITCT |
| | | VSGFSLTSYGVHWVRQSPGK |
| | | GLEWLGVIWSGGSTDYNAAFI |
| | | SRLSISKDTSKSQVFFKMNSLQ |
| | | PDDTAIYYCARNKDFYSNYDY |
| | | TMDYWGQGTSVTVSS |
| 48 | LC | MESQTQVFLSLLLWVSGTCGN |
| | | IMMTQSPSFLAVSAGEKVTLSC |
| | | KSSQSVLYSSNQKNFLAWYQ |
| | | QKPGQSPKLLIYWASTRESGV |
| | | PDRFTGSGSGTDFTLTISSVQA |
| | | EDLAVYYCHQYLSSYTFGGGT |
| | | KLEIK |
| 49 | HC | MAVLGLLFCLVTFPSCVLSEVQ |
| | | LVESGGGLVQPGGSLRLSCAA |
| | | SGFSLTTYGVHWVRQAPGKG |
| | | LEWVSVIWSGGDTDYNASFIS |
| | | RFTISRDNSKNTLYLQMNSLRA |
| | | EDTAVYYCARNKDYYTNYDFT |
| | | MDYWGQGTTVTVSS |
| 50 | LC | MESQTQVFLSLLLWVSGTCGD |
| | | IVMTQSPDSLAVSLGERATINC |
| | | KSSQSVLYSSNQKNFLAWYQ |
| | | QKPGQPPKLLIYWASTRESGV |
| | | PDRFSGSGSGTDFTLTISSLQA |
| | | EDVAVYYCHQYLSSYTFGQGT |
| | | KLEIK |
| 51 | HC | MDWTWVFLFLLSVTAGVHSQV |
| | | QLVQSGAEVKKPGSSVKVSCK |
| | | ASGYTFIDYWIEWVRQAPGQG |
| | | LEWMGEIFPGSGTINHNEKFK |
| | | DRVTITADKSTSTAYMELSSLR |
| | | SEDTAVYYCAREGLDYWGQG |
| | | TLVTVSS |
| 52 | LC | MEAPAQLLFLLLLWLPDTTGEI |
| | | VLTQSPATLSLSPGERATLSCS |
| | | ASSSVSYIYWYQQKPGQAPRL |
| | | LIYDTSTLASGIPARFSGSGSG |
| | | TDFTLTISSLEPEDFAVYYCQQ |
| | | WSRNPFTFGPGTKVDIK |
| 53 | HC | TVAAPSVFIFPPSDEQLKSGTA |
| | | SVVCLLNNFYPREAKVQWKVD |
| | | NALQSGNSQESVTEQDS |
| | | KDSTYSLSSTLTLSKADYEKHK |
| | | VYACEVTHQGLSSPVTKSFNR |
| | | GEC |

Anti-properdin antibodies described herein can be produced by using full-length properdin, properdin polypeptides, and/or using antigenic properdin epitope-bearing peptides, for example, a fragment of the properdin polypeptide. Properdin peptides and polypeptides can be isolated and used to generate antibodies as natural polypeptides, recombinant or synthetic recombinant polypeptides. All antigens useful for producing anti-properdin antibodies can be used to generate monovalent antibodies. Suitable monovalent antibody formats, and methods for producing them, are known in the art (e.g., WO 2007/048037 and WO 2007/059782, the entire contents of which are incorporated herein by reference).

The anti-properdin antibody may be a monoclonal antibody or derived from a monoclonal antibody. Suitable monoclonal antibodies to selected antigens may be prepared by known techniques (“Monoclonal Antibodies: A manual of techniques,” Zola (CRC Press, 1988); “Monoclonal Hybridoma Antibodies: Techniques and Applications,” Hurrell (CRC Press, 1982), the entire contents of which are incorporated herein by reference).

In other embodiments, the antibody may be a single-domain antibody, such as a V_{HH}. Such antibodies exist naturally, for example, in camelids and sharks (Saerens, D. et al., Curr. Opin. Pharmacol., 8:600-8, 2008). Camelid antibodies are described in, for example, U.S. Pat. Nos. 5,759,808; 5,800,988; 5,840,526; 5,874,541; 6,005,079; and 6,015,695, the entire contents of each of which are incorporated herein by reference. The cloned and isolated V_{HH} domain is a stable polypeptide that features the full antigen-binding capacity of the original heavy-chain antibody. V_{HH} domains, with their unique structural and functional properties, combine the advantages of conventional antibodies (high target specificity, high target affinity and low inherent toxicity) with important features of small molecule drugs (the ability to inhibit enzymes and access receptor clefts). Furthermore, they are stable, have the potential to be administered by means other than injection, are easier to manufacture, and can be humanized (U.S. Pat. Nos. 5,840,526; 5,874,541; 6,005,079, 6,765,087; EP 1589107; WO 97/34103; WO 97/49805; U.S. Pat. Nos. 5,800,988; 5,874,541 and 6,015,695, the entire contents of each of which are incorporated herein by reference). Such V_{HH} may have a polypeptide described in Table 3, below.

TABLE 3
Exemplary anti-properdin V_{HH}

| SEQ | |
|---|---|
| ID NO: | SEQUENCE |
| 31 | QVQVVESGGGLRQTGGSLRLSCTASGRIFEVNMMAWYRQAPGK |
| | QRELVAEISRVGTTVYADSVKGRFTISRDSAKNTVTLQMNSLK |
| | SEDTAVYYCNALQYDRYGGAEYWGQGTQVTVSS |
| 32 | QVQLAESGGGLVQAGDSLKLSCTASGRIFEVNMMAWYRQAPGK |
| | DRELVAEISRVGTTTYADSVKGRFTISRDSAKNTVTLQMNSLK |
| | SEDTAVYYCNALQYSRYGGAEYWGQGTQVTVSG |
| 33 | QVQLVESGGGLVQAGGSLRLSCAASGRISSIIHMAWYRQAPGK |
| | QRELVAEISRVGTTVYADSVKGRFTISRDDAKNTVTLQMNSLK |
| | PEDTAVYYCNALQYEKHGGADYWGQGTQVTVSG |
| 34 | QVQLVESGGGLRQTGESLRLSCTASGRIFEVNMMAWYRQAPGK |
| | QRELVAEISRVGTTTYADSVKGRFTISRDSAKNTVTLQMNSLK |
| | SEDTAVYYCNALQYDRYGGAEYWGQGTQVTVSG |

In some embodiments, where the anti-properdin binding domain has an exposed N-terminus, the N-terminal glutamine can convert into the cyclized pyro-glutamate. Such modifications are known in the art (see, e.g., Liu et al., The Journal of Biological Chemistry 286(13:11211-11217, 2011).

The V_{HH} may include one or more amino acid modifications. The amino acid modifications described herein include all amino acid modifications known in the art (see, e.g., Liu et al., The Journal of Biological Chemistry 286(13:11211-11217, 2011 and Manning et al., Pharmaceutical Research 27(4):544-575, 2010). In all contexts, known conversions of specific amino acids, e.g., during processing or purification of the fusion polypeptide, are to be included, e.g., conversion of an exposed N-terminal glutamine to pyro-glutamate.

Ia. Anti-Properdin Antibody Fragments and Derivatives

Some naturally occurring antibodies include two antigen binding domains and are therefore divalent. A number of smaller antigen binding fragments of naturally occurring antibodies have been identified following protease digestion. These include, for example, the “Fab fragment” (V_{L}-C_{L}-C_{H}1-V_{H}), “Fab′ fragment” (a Fab with the heavy chain hinge region), and “F(ab′)₂ fragment” (a dimer of Fab′ fragments joined by the heavy chain hinge region). Recombinant methods have been used to generate such fragments and to generate even smaller antibody fragments, e.g., those referred to as “single chain Fv” (variable fragment) or “scFv,” consisting of V_{L} and V_{H} joined by a synthetic peptide linker (V_{L}-linker-V_{H}). Fab fragments, Fab′ fragments and scFv fragments are monovalent for antigen binding, as they each include only one antigen binding domain including one V_{H}/V_{L} dimer. Even smaller monovalent antibody fragments are the dAbs, which include only a single immunoglobulin variable domain, e.g., V_{H} or V_{L}, that alone specifically binds antigen, i.e., without the need for a complementary V_{L} or V_{H} domain, respectively. A dAb binds antigen independently of other V domains; however, a dAb can be present in a homo- or hetero-multimer with other V_{H} or V_{L} domains where the other domains are not required for antigen binding by the dAb, i.e., where the dAb binds antigen independently of the additional V_{H} or V_{L} domains.

Ib. Linkers

In the present disclosure, a linker is used to join polypeptides or protein domains and/or associated non-protein moieties. In some embodiments, a linker is a linkage or connection between at least two polypeptide constructs, e.g., such that the two polypeptide constructs are joined to each other in tandem series (e.g., a monovalent antibody linked to a second polypeptide or monovalent antibody). A linker can attach the N-terminus or C-terminus of one antibody construct to the N-terminus or C-terminus of a second polypeptide construct.

A linker can be a simple covalent bond, e.g., a peptide bond, a synthetic polymer, e.g., a polyethylene glycol (PEG) polymer, or any kind of bond created from a chemical reaction, e.g., chemical conjugation. In the case that a linker is a peptide bond, the carboxylic acid group at the C-terminus of one protein domain can react with the amino group at the N-terminus of another protein domain in a condensation reaction to form a peptide bond. Specifically, the peptide bond can be formed from synthetic means through a conventional organic chemistry reaction well-known in the art, or by natural production from a host cell, wherein a polynucleotide sequence encoding the DNA sequences of both proteins, e.g., two antibody constructs, in tandem series can be directly transcribed and translated into a contiguous polypeptide encoding both proteins by the necessary molecular machineries, e.g., DNA polymerase and ribosome, in the host cell.

In the case that a linker is a synthetic polymer, e.g., a PEG polymer, the polymer can be functionalized with reactive chemical functional groups at each end to react with the terminal amino acids at the connecting ends of two proteins.

In the case that a linker (except peptide bond mentioned above) is made from a chemical reaction, chemical functional groups, e.g., amine, carboxylic acid, ester, azide, or other functional groups commonly used in the art, can be attached synthetically to the C-terminus of one protein and the N-terminus of another protein, respectively. The two functional groups can then react to through synthetic chemistry means to form a chemical bond, thus connecting the two proteins together. Such chemical conjugation procedures are routine for those skilled in the art.

A linker between two peptide constructs can be, for example, an amino acid linker including from 1-200 (e.g., 1-4, 1-10, 1-20, 1-30, 1-40, 2-10, 2-12, 2-16, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200) amino acids. Suitable peptide linkers are known in the art, and include, for example, peptide linkers containing flexible amino acid residues such as glycine and serine. In certain embodiments, a linker can contain single motifs or multiple different or repeating motifs.

In some embodiments, the linker is a poly-glycine linker. In some embodiments, the poly-glycine linker includes the sequence GGGGE (SEQ ID NO: 5).

Ic. Bispecific Constructs

The disclosure also features bispecific constructs where two antigen binding polypeptides are linked. Such bispecific constructs may include an anti-properdin binding polypeptide (e.g., a monovalent antibody) connected by a linker to a second polypeptide (e.g., a second monovalent antibody). The second polypeptide can enhance in vivo stability of the bispecific construct. In some embodiments, the second polypeptide is an albumin binding molecule, an albumin binding peptide, an anti-albumin antibody (e.g., a monovalent antibody), an anti-human serum albumin or a modified form thereof. Albumin binding peptides are known in the art and are described, for example, in WO 2007/106120 (see Tables 1 to 9) and Dennis et al., 2002, J Biol. Chem. 277: 35035-35043, the disclosures of which are hereby incorporated by reference.

In some embodiments, the second polypeptide is a Fc domain that enhances in vivo stability of the construct.

In some embodiments, a monovalent anti-properdin antibody is linked to a monovalent anti-albumin antibody. The monovalent anti-properdin antibody may be linked by its N-terminus or C-terminus to the N-terminus or C-terminus of the monovalent anti-albumin antibody.

Id. Exemplary Anti-Properdin Antibodies In some embodiments, the anti-properdin/anti-human serum albumin bispecific construct comprises six CDR sequences of SEQ ID NO: 6 (SEQ ID NOS:2, 3, and 4, and 55, 56, and 57).

In some embodiments, the anti-human serum albumin component of a bispecific construct comprises the CDR sequences GRPVSNYA (SEQ ID NO: 55), INWQKTAT (SEQ ID NO: 56), and AAVFRVVAPKTQYDYDY (SEQ ID NO: 57).

In some embodiments, the anti-properdin bi-specific construct comprises the sequence of:

(SEQ ID NO: 6)
QVQLVESGGGLVKPGGSLRLSCAASGRPVSNYAAAWFRQAPGKEREF
VSAINWQKTATYADSVKGRFTISRDNAKNSLYLQMNSLRAEDTAVYY
CAAVFRVVAPKTQYDYDYWGQGTLVTVSSGGGGEGGGGEGGGGEVQL
LESGGGLVQPGGSLRLSCAASGRISSIIHMAWFRQAPGKERELVSEI
SRVGTTVYADSVKGRFTISRDNSKNTLYLQMNSLKPEDTAVYYCNAL
QYEKHGGADYWGQGTLVTVSS.

In some embodiments, the anti-properdin bi-specific construct comprises the sequence of:

(SEQ ID NO: 35)
EVQLVESGGGLVKPGGSLRLSCAASGRPVSNYAAAWFRQAPGKEREF
VSAINWQKTATYADSVKGRFTISRDNAKNSLYLQMNSLRAEDTAVYY
CAAVFRVVAPKTQYDYDYWGQGTLVTVSSGGGGSGGGGSGGGGSLEV
QLVESGGGLVQAGGSLRLSCAASGRISSIIHMAWYRQAPGKQRELVA
EISRVGTTVYADSVKGRFTISRDDAKNTVTLQMNSLKPEDTAVYYCN
ALQYEKHGGADYWGQGTQVTVSS.

In some embodiments, the anti-properdin bi-specific construct comprises the sequence of:

(SEQ ID NO: 36)
EVQLVESGGGLVKPGGSLRLSCAASGRPVSNYAAAWFRQAPGKEREF
VSAINWQKTATYADSVKGRFTISRDNAKNSLYLQMNSLRAEDTAVYY
CAAVFRVVAPKTQYDYDYWGQGTLVTVSSGGGGSGGGGSGGGGSEVQ
LLESGGGLVQPGGSLRLSCAASGRISSIIHMAWFRQAPGKERELVSE
ISRVGTTVYADSVKGRFTISRDNSKNTLYLQMNSLKPEDTAVYYCNA
LQYEKHGGADYWGQGTLVTVSS.

In some embodiments, the anti-properdin bi-specific construct comprises the sequence of: A

(SEQ ID NO: 37)
EVQLVESGGGLVKPGGSLRLSCAASGRPVSNYAAAWFRQAPGKEREF
VSAINWQKTATYADSVKGRFTISRDNAKNSLYLQMNSLRAEDTAVYY
CAAVFRVVAPKTQYDYDYWGQGTLVTVSSGGGGDGGGGDGGGGEVQL
VESGGGLVQAGGSLRLSCAASGRISSIIHMAWYRQAPGKQRELVAEI
SRVGTTVYADSVKGRFTISRDDAKNTVTLQMNSLKPEDTAVYYCNAL
QYEKHGGADYWGQGTQVTVSS.

In some embodiments, the anti-properdin bi-specific construct comprises the sequence of:

(SEQ ID NO: 38)
EVQLVESGGGLVKPGGSLRLSCAASGRPVSNYAAAWFRQAPGKEREF
VSAINWQKTATYADSVKGRFTISRDNAKNSLYLQMNSLRAEDTAVYY
CAAVFRVVAPKTQYDYDYWGQGTLVTVSSGGGGEGGGGEGGGGEVQL
VESGGGLVQAGGSLRLSCAASGRISSIIHMAWYRQAPGKQRELVAEI
SRVGTTVYADSVKGRFTISRDDAKNTVTLQMNSLKPEDTAVYYCNAL
QYEKHGGADYWGQGTQVTVSS.

In some embodiments, the anti-properdin bi-specific construct comprises the sequence of:

(SEQ ID NO: 39)
EVQLVESGGGLVKPGGSLRLSCAASGRPVSNYAAAWFRQAPGKEREF
VSAINWQKTATYADSVKGRFTISRDNAKNSLYLQMNSLRAEDTAVYY
CAAVFRVVAPKTQYDYDYWGQGTLVTVSSGGGGSGGGGSGGGGSEVQ
LVESGGGLVQPGGSLRLSCAASGRISSIIHMAWVRQAPGKQRELVSE
ISRVGTTVYADSVKGRFTISRDNSKNTLYLQMNSLRAEDTAVYYCNA
LQYEKHGGADYWGQGTLVTVSS.

In some embodiments, the anti-properdin bi-specific construct comprises the sequence of:

(SEQ ID NO: 40)
EVQLVESGGGLVKPGGSLRLSCAASGRPVSNYAAAWFRQAPGKEREF
VSAINWQKTATYADSVKGRFTISRDNAKNSLYLQMNSLRAEDTAVYY
CAAVFRVVAPKTQYDYDYWGQGTLVTVSSGGGGDGGGGDGGGGEVQL
LESGGGLVQPGGSLRLSCAASGRISSIIHMAWFRQAPGKERELVSEI
SRVGTTVYADSVKGRFTISRDNSKNTLYLQMNSLKPEDTAVYYCNAL
QYEKHGGADYWGQGTLVTVSS.

In some embodiments, the anti-properdin bi-specific construct comprises the sequence of:

(SEQ ID NO: 41)
EVQLVESGGGLVKPGGSLRLSCAASGRPVSNYAAAWFRQAPGKEREF
VSAINWQKTATYADSVKGRFTISRDNAKNSLYLQMNSLRAEDTAVYY
CAAVFRVVAPKTQYDYDYWGQGTLVTVSSGGGGEGGGGEGGGGEVQL
LESGGGLVQPGGSLRLSCAASGRISSIIHMAWFRQAPGKERELVSEI
SRVGTTVYADSVKGRFTISRDNSKNTLYLQMNSLKPEDTAVYYCNAL
QYEKHGGADYWGQGTLVTVSS.

In some embodiments, the anti-properdin bi-specific construct comprises the sequence of:

(SEQ ID NO: 42)
EVQLVESGGGLVKPGGSLRLSCAASGRPVSNYAAAWFRQAPGKEREF
VSAINWQKTATYADSVKGRFTISRDNAKNSLYLQMNSLRAEDTAVYY
CAAVFRVVAPKTQYDYDYWGQGTLVTVSSGGGGDGGGGDGGGGEVQL
VESGGGLVQPGGSLRLSCAASGRISSIIHMAWVRQAPGKQRELVSEI
SRVGTTVYADSVKGRFTISRDNSKNTLYLQMNSLRAEDTAVYYCNAL
QYEKHGGADYWGQGTLVTVSS.

In some embodiments, where the anti-properdin binding domain or the anti-human serum albumin binding domain has an exposed N-terminus, the N-terminal glutamine can convert into the cyclized pyro-glutamate. Such modifications are known in the art (see, e.g., Liu et al., The Journal of Biological Chemistry 286(13:11211-11217, 2011).

The fusion protein may include one or more amino acid modifications. The amino acid modifications described herein include all amino acid modifications known in the art (see, e.g., Liu et al., The Journal of Biological Chemistry 286(13:11211-11217, 2011 and Manning et al., Pharmaceutical Research 27(4):544-575, 2010). In all contexts, known conversions of specific amino acids, e.g., during processing or purification of the fusion polypeptide, are to be included, e.g., conversion of an exposed N-terminal glutamine to pyro-glutamate.

In some embodiments, the anti-properdin by specific construct is encoded by the following sequence:

(SEQ ID NO: 54)
CAGGTGCAGCTGGTGGAAAGCGGCGGAGGCCTGGTCAAGCCTGGCGG
CAGCCTGAGACTGAGCTGTGCCGCCAGCGGCAGACCCGTGTCCAATT
ACGCCGCTGCCTGGTTCCGGCAGGCCCCTGGCAAAGAGAGAGAGTTC
GTCAGCGCCATCAACTGGCAGAAAACCGCCACCTACGCCGACAGCGT
GAAGGGCCGGTTCACCATCAGCCGGGACAACGCCAAGAACAGCCTGT
ACCTGCAGATGAACTCCCTGCGGGCCGAGGACACCGCCGTGTACTAC
TGCGCCGCTGTGTTCCGGGTGGTGGCCCCCAAGACCCAGTACGACTA
CGATTACTGGGGCCAGGGCACCCTGGTCACCGTGTCATCTGGCGGAG
GGGGAGAAGGCGGGGGAGGGGAAGGGGGAGGCGGCGAAGTCCAGCTG
CTGGAATCTGGGGGGGGACTGGTGCAGCCAGGCGGCTCCCTCAGACT
GTCTTGCGCCGCCTCCGGCCGGATCAGCAGCATCATCCACATGGCCT
GGTTTAGACAGGCTCCCGGAAAAGAACGCGAGCTGGTGTCCGAGATC
TCCAGAGTGGGCACCACCGTGTATGCCGACTCCGTGAAAGGCAGATT
CACAATCTCCCGCGACAACAGCAAGAATACTCTGTATCTCCAGATGA
ATAGCCTGAAGCCCGAAGATACAGCCGTCTACTATTGCAACGCCCTG
CAGTACGAGAAGCACGGCGGAGCCGACTATTGGGGACAGGGAACACT
CGTGACAGTGTCTAGCTGATGA.

### II. Nucleic Acids

IIa. Inhibitory RNA

In some embodiments, the complement inhibitor is an inhibitory RNA molecule, e.g., that acts by way of the RNA interference (RNAi) pathway. An inhibitory RNA molecule can decrease the expression level (e.g., protein level or mRNA level) of a complement protein (e.g., complement C3, factor B, or properdin). For example, an inhibitory RNA molecule includes a siRNA, shRNA, and/or a miRNA that targets full-length complement C3, factor B, or properdin. An siRNA is a double-stranded RNA molecule that typically has a length of about 19-25 base pairs. An shRNA is an RNA molecule containing a hairpin turn that decreases expression of target genes via RNAi. shRNAs can be delivered to cells in the form of plasmids (e.g., viral or bacterial vectors), by transfection, electroporation, or transduction. A microRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. In some embodiments, the inhibitory RNA molecule decreases the level and/or activity of a complement proteins functions. In other embodiments, the inhibitory RNA molecule decreases the level and/or activity of an inhibitor of a positive regulator of function.

An inhibitory RNA molecule can be modified, e.g., to contain modified nucleotides, e.g., 2′-fluoro,2′-o-methyl, 2′-deoxy, unlocked nucleic acid, 2′-hydroxy, phosphorothioate, 2′-thiouridine, 4′-thiouridine, or 2′-deoxyuridine. Without being bound by a particular theory, it is believed that certain modification can increase nuclease resistance and/or serum stability or decrease immunogenicity.

In some embodiments, the inhibitory RNA molecule decreases the level and/or activity or function of a complement protein (e.g., complement C3, factor B, or properdin). In some embodiments, the inhibitory RNA molecule inhibits expression of a complement protein (e.g., complement C3, factor B, or properdin). In other embodiments, the inhibitory RNA molecule increases degradation of a complement protein (e.g., complement C3, factor B, or properdin). The inhibitory RNA molecule can be chemically synthesized or transcribed in vitro. The making and use of inhibitory therapeutic agents based on non-coding RNA such as ribozymes, RNAase P, siRNAs, and miRNAs are also known in the art, for example, as described in Sioud, RNA Therapeutics: Function, Design, and Delivery (Methods in Molecular Biology). Humana Press 2010.

IIb. Antisense

In one approach, the invention provides a single-stranded oligonucleotide having a nucleobase sequence with at least 6 contiguous nucleobases complementary to an equal-length portion within a complement protein (e.g., complement C3, factor B, or properdin) target nucleic acid. This approach is typically referred to as an antisense approach. Without wishing to be bound by theory, this approach involves hybridization of an oligonucleotide to a target nucleic acid (e.g., properdin pre-mRNA transcript 1, or transcript 2, respectively), followed by ribonuclease h (RNase H) mediated cleavage of the target nucleic acid. Alternatively, and without wishing to be bound by theory, this approach involves hybridization of an oligonucleotide to a target nucleic acid (e.g., complement C3, factor B, or properdin pre-mRNA transcript 1, or transcript 2, respectively), thereby sterically blocking the target nucleic acid from binding cellular post-transcription modification or translation machinery and thus preventing the translation of the target nucleic acid. In some embodiments, the single-stranded oligonucleotide may be delivered to a patient as a double stranded oligonucleotide, where the oligonucleotide is hybridized to another.

### III. Aptamer

In some embodiments, the complement pathway component inhibitor may be an aptamer. Any suitable aptamer may be used. General descriptions of aptamer are described in Bock L C et al., Nature 355 (6360): 564-6 (1992); Hoppe-Seyler F, Butz K “Peptide aptamers: powerful new tools for molecular medicine”. J Mol Med. 78 (8): 426-30 (2000); Cohen B A, Colas P, Brent R. “An artificial cell-cycle inhibitor isolated from a combinatorial library”. Proc Natl Acad Sci USA. 95 (24): 14272-7 (1998).

Aptamers are isolated nucleic acid molecules that bind with high specificity and affinity to some target, such as a protein (e.g., a factor that activates the CAP), by an interaction other than Watson-Crick base pairing. Aptamers are nucleic acid-based molecules, but there are fundamental differences between aptamers and other nucleic acid molecules, such as genes and mRNAs. In the latter case, the nucleic acid structure encodes information by its linear base sequence, and thus this sequence is important for information storage function. In contrast, aptamer function is dependent on the specific secondary/tertiary structure rather than the conserved linear base sequence, based on the binding of the specific target molecule. That is, the aptamer is a non-coding sequence. Any codeability that an aptamer can have is quite accidental and plays no role in the binding of an aptamer to its cognate target. Thus, aptamers that bind to the same target and even to the same site on the target may share a similar linear base sequence, but most do not.

In some embodiments, the aptamer is comprises a series of nucleic acid aptamers of about 15 to about 60 nucleotides in length that bind specifically to a CAP factor and modulate the activity of the CAP factor.

These aptamers may include modifications as described herein including, e.g., conjugation to lipophilic or high molecular weight compounds (e.g., PEG), incorporation of a capping moiety, incorporation of modified nucleotides, and modifications to the phosphate back bone.

In some embodiments, the aptamer is an anti-C5 aptamer, e.g., Avacincaptad Pegol (ARC-1905; CAS #1491144-00-3 and FDA Drug #K86ENL1215). In some embodiments, the aptamer is Factor B-binding aptamer that also inhibits C3 convertase. Representative examples, e.g., SL1102 and SL1103, are provided in. Xu et al. (J Immunol., 206(4): 861-873, 2021; PMID: 33419768).

### IV. Small Molecules

In some embodiments, the complement pathway component inhibitor may be a small molecule.

Small molecules are molecules, typically with a molecular weight less than about 1000 Daltons, or in some embodiments, less than about 500 Daltons, wherein the molecule is capable of modulating, to some measurable extent, an activity of a target molecule. Exemplary small molecules such as peptides and small molecule inhibitors. Small molecules, such as small molecule inhibitors, may be selected based on the efficacy and specificity.

In some embodiments, the inhibitor of complement pathway comprises a factor D inhibitor. Representative oral factor D inhibitors are disclosed in WO2015130838 and U.S. Pat. No. 9,732,103, the disclosures in which are incorporated by reference in their entirety. Other representative factor D inhibitors are disclosed in WO2017035353 and U.S. Pat. No. 10,011,612, the disclosures in which are incorporated by reference in their entirety. Still other representative factor D inhibitors are disclosed in WO2018160889 and US Pub. No. 2020/0071301, the disclosures in which are incorporated by reference in their entirety.

In some embodiments, the Factor D inhibitor useful in the therapeutic methods of the present application comprises danicopan (compound 1 or a salt thereof):

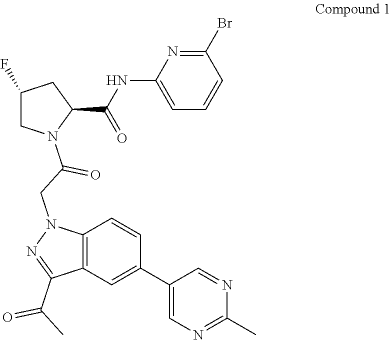 [SMILES: CC(=O)c1nn(CC(=O)N2C[C@H](F)C[C@H]2C(=O)Nc2cccc(Br)n2)c2ccc(-c3cnc(C)nc3)cc12]

In some embodiments, the Factor D inhibitor useful in the therapeutic methods of the present application comprises vermicopan (compound 2 or a salt thereof):

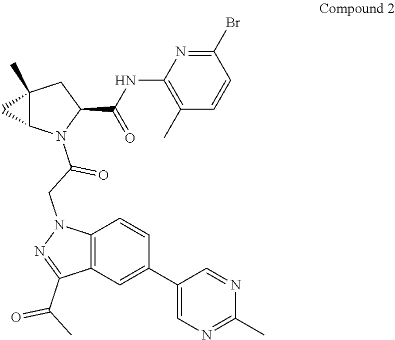 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]

In some embodiments, the Factor D inhibitor useful in the therapeutic methods of the present application comprises a factor D inhibitor of compound 3 or a salt thereof:

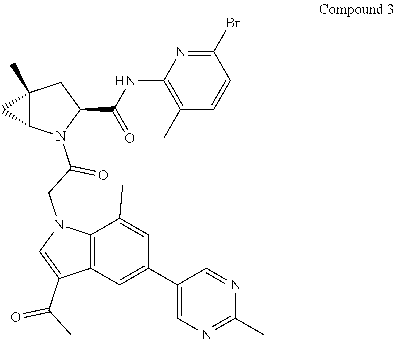 [SMILES: CC(=O)c1cn(CC(=O)N2[C@H](C(=O)Nc3nc(Br)ccc3C)C[C@@]3(C)C[C@@H]23)c2c(C)cc(-c3cnc(C)nc3)cc12]

In some embodiments, the Factor D inhibitor useful in the therapeutic methods of the present application comprises a factor D inhibitor of compound 4 or a salt thereof:

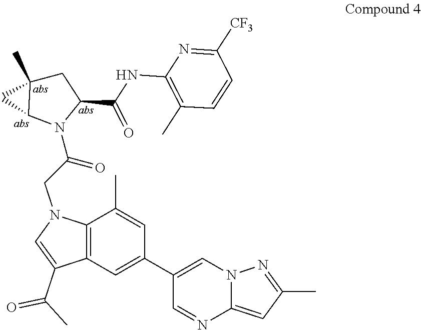 [SMILES: CC(=O)c1cn(CC(=O)N2[C@H](C(=O)Nc3nc(C(F)(F)F)ccc3C)C[C@@]3(C)C[C@@H]23)c2c(C)cc(-c3cnc4cc(C)nn4c3)cc12]

In some embodiments, the salt is hydrochloride. In some embodiments, the salt is any pharmaceutically acceptable salt, for example tosylate, sulphate, and the like.

### V. Peptides

In some embodiments the inhibitor of the CAP is a peptide, e.g., a cyclic peptide, inhibitor of an AP pathway component (e.g., complement factor C3). Known peptide inhibitors of complement factor C3 include Compstatin and derivatives thereof.

A peptide inhibitor may be any peptide that binds specifically to protein in the complement alternative pathway (e.g., complement factor C3), or a protein that inhibits or neutralizes the function of a protein thereof. Peptide inhibitors may be chemically synthesized using known peptide synthesis methodology or may be prepared and purified using recombinant technology. Peptide inhibitors may be identified without undue experimentation using well known techniques. In this regard, it is noted that techniques for screening peptide libraries for peptides that are capable of specifically binding to a CAP polypeptide target are well known in the art.

### Exemplary Therapeutic Approaches

Exemplary therapeutic approaches to treatment of SCD, BT, or sickle cell BT are provided in Table 4 and in the paragraphs below:

Included below are exemplary approaches to treating patients with SCD, BT, or sickle cell BT.

TABLE 4
Therapeutic Approaches to treating SCD, BT, or sickle cell BT by modulating the
complement system

| Target | Therapeutic | Developer/Distributor | Modality |
|---|---|---|---|
| C1q | Anti-C1q mAb | Annexon | Antibody |
| C1 | C1-INH (BERINERT, | CSL Behring, Salix Pharma, | Polypeptide/protein |
| | RUCONEST, CYNRIZE) | Shire, respectively | |
| C1s | Anti-C1s mAbBIVV020 or | True North Therapeutics | Antibody |
| | activated anti-C1s Ab | Sanofi | |
| | Sutimlimab (BIVV009 or | Bioverativ, a Sanofi | Antibody |
| | TNT009) | Company | |
| | C1s peptide | Ra Pharma | Peptide |
| C2 | PRO-02 mAb | Prothix BV | Antibody |
| MASP-2 | Narsoplimab (α-MASP-2 | Omeros | Antibody |
| | mAb) | | |
| MASP-3 | OMS906 (α-MASP-3 mAb) | Omeros | Antibody |
| Factor D | Anti-FD mAb | Genentech | Antibody |
| | (Lampalizumab) | | |
| | ALXN 2040 (ACH-4471); | Achillion | Small molecule |
| | ALXN 2050 (ACH-5228); or | | |
| | third generation FD inhibitor | | |
| | BCX9930 | Biocryst | Small molecule |
| | Various (see, Maibaum et | Novartis | Small molecules |
| | al. (Nat Chem Biol. 2016 | | |
| | 12(12): 1105-10)) | | |
| Factor B | Factor B siRNA IONIS-FB- | Ionis | siRNA/nucleic acid |
| | LRX | | |
| | Anti-Factor B mAb | Novelmed/Alexion, | Antibody |
| | | AstraZeneca Rare Disease | |
| | Iptacopan (LNP023; CAS: | Novartis | Small molecule |
| | 1644670-37-0; FDA Drug | | |
| | No. 8E05T07Z6W) | | |
| Properdin | Anti-Properdin mAb | Novelmed | Antibody |
| | CLG561 Mab | Novartis | Antibody |
| Properdin | ALXN1820 | Alexion, AstraZeneca Rare | Bispecific minibody |
| | | Disease | |
| Factor H | AMY-201, ‘mini-factor H’ | Amyndas | Polypeptide |
| C3/C5 | Compstatin/Derivative - | Apellis | Peptide |
| Convertases | APL2, APL9 | | |
| | Compstatin/Derivative - | Amyndas | Peptide |
| | AMY-101 | | |
| | SCR1/TP10 | Celldex | Polypeptide |
| | Mini-FH | Amyndas | Polypeptide |
| | Mirococept (APT070) | AdProTech | Polypeptide conjugate |
| | CR2-Factor H/TT30 | Alexion, AstraZeneca Rare | Fusion protein |
| | | Disease | |
| C3a/C3aR | Various | Both experimental and | Mostly small |
| | | therapeutic | molecules, e.g., |
| | | | SB290157 |
| | | | (Calbiochem) |
| C6 | Anti-C6 mAb | Regenesance | Antibody |
| | C6 Anti-sense RNA | | Antisense nucleotide |
| | CP010 | Complement | Antibody |
| | | Pharma/Alexion, | |
| | | AstraZeneca Rare Disease | |
| CD59 | Adeno associated vector | Hemera | Gene therapy |
| | (AAV) CAGsCD59 | | |
| | (HMR59) | | |

By way of example, the disclosure relates to the following methods for monitoring efficacy of therapy of SCD (e.g., sickle cell anemia, BT, or sickle BT):
(A) A method for the treatment of SCD, BT, or sickle cell BT in a subject with an anti-C1q monoclonal antibody.
(B) A method for the treatment of SCD, BT, or sickle cell BT in a subject with a C1-INH (e.g., BERINERT, RUCONEST, CYNRIZE).
(C)(1) A method for the treatment of SCD, BT, or sickle cell BT in a subject with an anti-C1s monoclonal antibody (e.g., BIVV020 or activated anti-C1s antibody).
(C)(2) A method for the treatment of SCD, BT, or sickle cell BT in a subject with a C1s peptide. (D) A method for the treatment of SCD, BT, or sickle cell BT in a subject with an anti-C2 monoclonal antibody (e.g., PRO-02).
(E) A method for the treatment of SCD, BT, or sickle cell BT in a subject with an anti-MASP-2 monoclonal antibody (e.g., Narsoplimab).
(F) A method for the treatment of SCD, BT, or sickle cell BT in a subject with an anti-MASP-3 monoclonal antibody (e.g., OMS906).
(G) A method for the treatment of SCD, BT, or sickle cell BT in a subject with an anti-Factor D (FD) monoclonal antibody (e.g., lampalizumab).
(H) A method for the treatment of SCD, BT, or sickle cell BT in a subject with an oral, small molecule Factor D (FD) inhibitor (e.g., danicopan (ALXN2040 or ACH-4471) or vermicopan (ALXN2050 or ACH-5228) or a third generation FD inhibitor (compound 3)).
(I) A method for the treatment of SCD, BT, or sickle cell BT in a subject with a small molecule Factor D (FD) inhibitor (e.g., BCX9930 or FD inhibitors in U.S. Pat. No. 9,388,199, which is incorporated by reference herein).
(J) A method for the treatment of SCD, BT, or sickle cell BT in a subject with a Factor B (FB) inhibitor (e.g., Factor B siRNA IONIS-FB-L_{RX} or α-FB monoclonal antibody).
(K) A method for the treatment of SCD, BT, or sickle cell BT in a subject with a Factor B (FB) inhibitor (LNP023).
(L) A method for the treatment of SCD, BT, or sickle cell BT in a subject with anti-properdin (Factor P) monoclonal antibody (e.g., CLG561) or bispecific antibody (e.g., ALXN1820).
(M) A method for the treatment of SCD, BT, or sickle cell BT in a subject with a Factor H (FH) modulator (e.g., mini-factor H, AMY-201, or CR2-Factor H/TT30).
(N) A method for the treatment of SCD, BT, or sickle cell BT in a subject with C3 inhibitor selected from a compstatin or a derivative thereof (e.g., APL2, APL9, or AMY-101), sCR1/TP10, or Mirococept.
(O)(1) A method for the treatment of SCD, BT, or sickle cell BT in a subject with nomacopan (Coversin; rVA576).
(O)(2) A method for the treatment of SCD, BT, or sickle cell BT in a subject with Zilucoplan (RA101495).
(P)(1) A method for the treatment of SCD, BT, or sickle cell BT in a subject with Avacopan (CCX-168).
(P)(2) A method for the treatment of SCD, BT, or sickle cell BT in a subject with an anti-C5a monoclonal antibody (e.g., olendalizumab (ALXN1007) or BDB-001 or IFX2)).
(Q)(1) A method for the treatment of SCD, BT, or sickle cell BT in a subject with a complement C6 inhibitor selected from anti-C6 monoclonal antibody and C6 anti-sense RNA.
(Q)(2) A method for the treatment of SCD, BT, or sickle cell BT in a subject with a complement C6 inhibitor CP010.
(R) A method for the treatment of SCD, BT, or sickle cell BT in a subject with an adeno associated vector (AAV) encoding soluble CD59 (HMR59).

### Delivery

### I. Viral Vectors for Expression of Therapeutic Complement Protein Inhibitors

Viral genomes provide a rich source of vectors that can be used for the efficient delivery of exogenous genes into a mammalian cell (e.g., sickle cells). Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996)). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

Ia. Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral complimentary DNA (cDNA) deprived of all open reading frames, but maintaining the sequences required for replication, encapsidation, and expression, in which the sequences to be expressed are inserted.

Ib. Adeno-Associated Viral Vectors

Nucleic acids of the compositions and methods described herein may be incorporated into recombinant adeno-associated virus (rAAV) vectors and/or virions in order to facilitate their introduction into a cell (e.g., a sickle cell). rAAV vectors useful in the compositions and methods described herein are recombinant nucleic acid constructs (e.g., nucleic acids capable of expression in sickle cells) that include (1) a heterologous sequence to be expressed and (2) viral sequences that facilitate integration and expression of the heterologous genes. The viral sequences may include those sequences of AAV that are required in cis for replication and packaging (e.g., functional inverted terminal repeat sequences (ITR)) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. Methods for using rAAV vectors are described, for example, in Tai et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

### Pharmaceutical Compositions

The CAP inhibitor (e.g., antibodies, small molecules, nucleic acid molecules, peptides, and aptamers) described herein can be formulated, for example, into pharmaceutical compositions for administration to a patient, such as a human patient exhibiting or at risk of developing SCD, BT, or sickle cell BT, in a biologically compatible form suitable for administration in vivo. A pharmaceutical composition containing, for example, a complement protein inhibitor described herein, such as an interfering RNA molecule, typically includes a pharmaceutically acceptable diluent or carrier. A pharmaceutical composition may include (e.g., consist of), e.g., a sterile saline solution and a nucleic acid. The sterile saline is typically a pharmaceutical grade saline. A pharmaceutical composition may include (e.g., consist of), e.g., sterile water and a nucleic acid. The sterile water is typically a pharmaceutical grade water. A pharmaceutical composition may include (e.g., consist of), e.g., phosphate-buffered saline (PBS) and a nucleic acid. The sterile PBS is typically a pharmaceutical grade PBS.

In certain embodiments, pharmaceutical compositions include one or more CAP inhibitors and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, complement protein inhibitors may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions including a CAP inhibitor encompass any pharmaceutically acceptable salts of the inhibitor, esters of the inhibitor, or salts of such esters. In certain embodiments, pharmaceutical compositions including a complement protein inhibitor, upon administration to a subject (e.g., a human), are capable of providing (directly or indirectly) the biologically active metabolite or residue thereof. Accordingly, for example, the disclosure is also drawn to pharmaceutically acceptable salts of inhibitors, prodrugs, pharmaceutically acceptable salts of such prodrugs, and other bioequivalents. Suitable pharmaceutically acceptable salts include, but are not limited to, sodium and potassium salts. In certain embodiments, prodrugs include one or more conjugate group attached to a complement protein inhibitor, wherein the conjugate group is cleaved by endogenous nucleases within the body.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions include a delivery system. Examples of delivery systems include, but are not limited to, liposomes and emulsions. Certain delivery systems are useful for preparing certain pharmaceutical compositions including those including hydrophobic compounds. In certain embodiments, certain organic solvents such as dimethylsulfoxide are used.

In certain embodiments, pharmaceutical compositions include one or more tissue-specific delivery molecules designed to deliver the one or more pharmaceutical agents of the present disclosure to specific tissues or cell types. For example, in certain embodiments, pharmaceutical compositions include liposomes coated with a tissue-specific antibody.

In certain embodiments, pharmaceutical compositions include a co-solvent system. Certain of such co-solvent systems include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical composition is prepared for administration by injection (e.g., intravenous). In certain of such embodiments, a pharmaceutical composition includes a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

### Kits

The compositions described herein can be provided in a kit for use in treating SCD, BT, or sickle cell BT. The kit may include one or more AP inhibitors, specifically, properdin inhibitors, as described herein. The kit can include a package insert that instructs a user of the kit, such as a physician, to perform any one of the methods described herein. The kit may optionally include a syringe or other device for administering the composition. In some embodiments, the kit may include one or more additional therapeutic agents.

### EXAMPLES

The following are examples of the methods of the disclosure. It is understood that various other embodiments may be practiced, given the general description provided above.

### Example 1. Efficacy of Inhibition of Complement Activation in Hypoxia-Induced Vaso-Occlusion Crisis

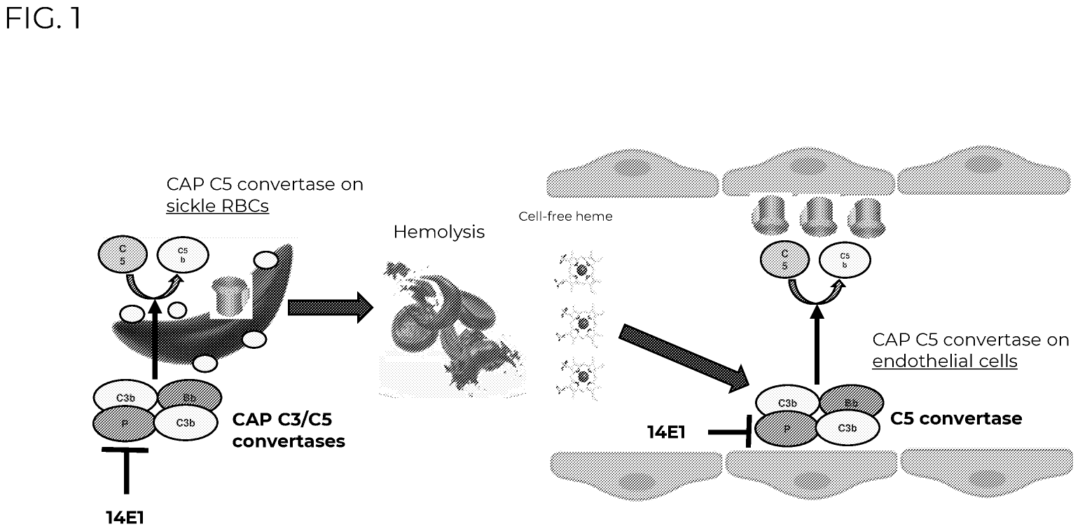

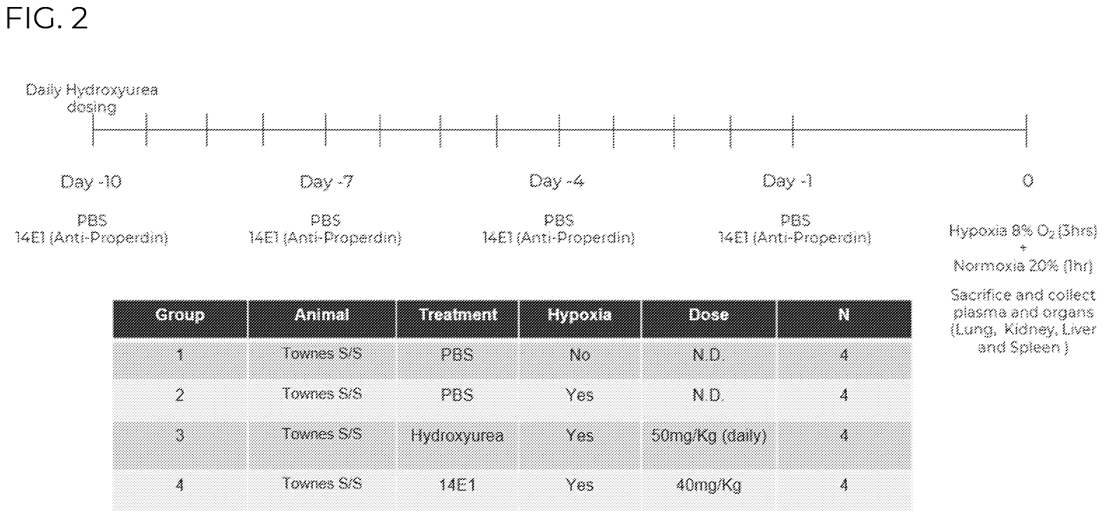

To demonstrate the efficacy of inhibition of complement activation (FIG. 1) in VOC, Townes SS mice are divided into four groups and prophylactically treated with either PBS (vehicle) or 14E1 (mouse anti-properdin) four times from ten days before hypoxia treatment. A representative experimental setup is provided in FIG. 2. Animals were sacrificed after hypoxia treatment followed by one hour resting in normoxic condition. In one of the vehicle-treated group, animals were not exposed to hypoxic condition and continuously maintained in normoxic condition throughout the experiment and serve as a baseline. Upon euthanasia, blood samples and critical organs were harvested from animals to measure the level of complement deposition on RBCs, intravascular hemolysis and the severity of vaso-occlusions.

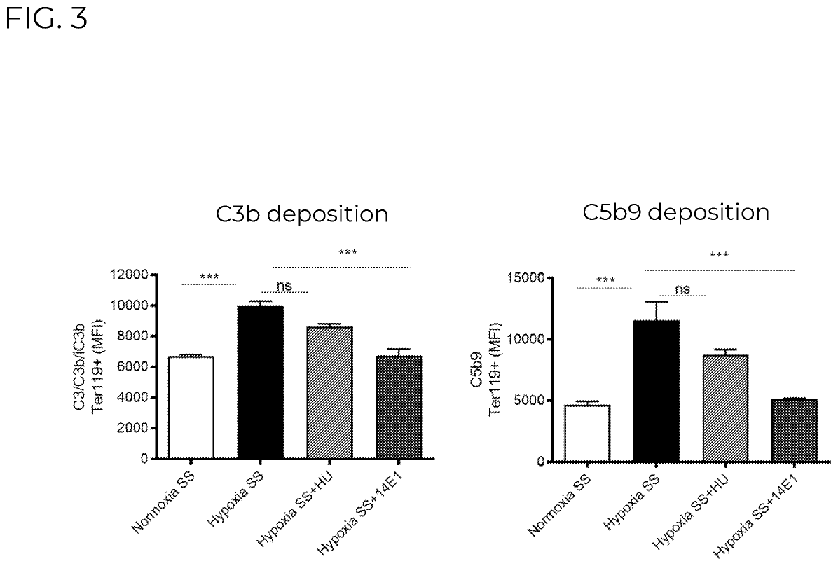

Flow cytometry-based RBC analyses revealed increased complement fragment deposition, both C5b9 and C3 in SS RBCs exposed to hypoxic condition (FIG. 3). However, pretreatment with anti-properdin monoclonal antibody (14E1) prevented increase in C5b9 deposition. The increase in C3b deposition in SS RBC under hypoxic conditions was prevented by anti-properdin MAb. This observation is consistent with the notion of properdin being a key component of both CAP C3 and C5 convertases and prevention of C3 opsonization of PNH RBCs by anti-properdin mAb treatment.

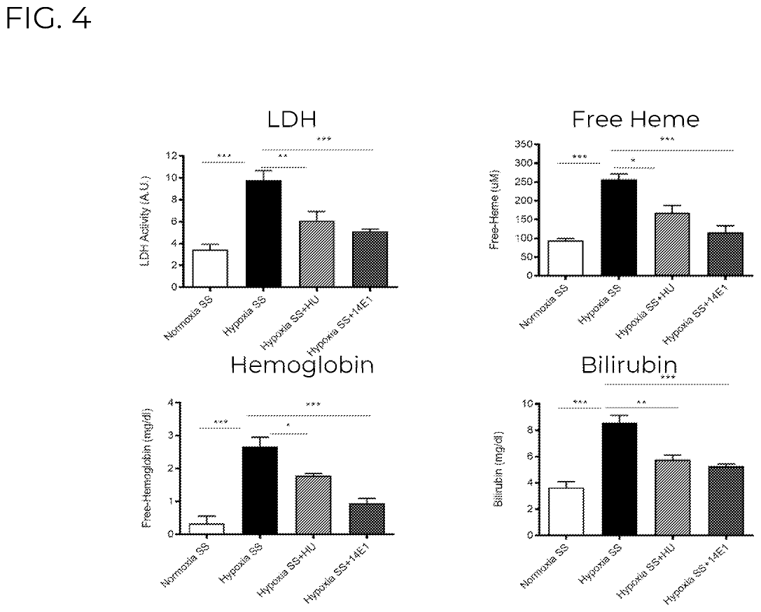

Next, changes in the level of intravascular hemolysis were determined by various assays including plasma lactate dehydrogenase (LDH) activity, free heme and free hemoglobin and total bilirubin level. Exposure of the SCD animals to hypoxic condition triggered intravascular hemolysis (IVH) which is effectively prevented by pretreatment with anti-properdin MAb (FIG. 4). IVH was assayed using recognized markers such as LDH, bilirubin, free hemoglobin and free heme. These data of inhibition of intravascular hemolysis in SCD mice by anti-complement therapy has clear implications in SCD patients as these hemolysis biomarkers have been well validated in SCD patients. Elevated LDH activity is associated with mortality and morbidity of SCD patients at the steady state (Kato et al. 2006) or during painful VOC events (Ballas and Marcolina 2006). Moreover, a positive correlation between LDH activity and the severity of pain has been reported among children during VOC (Najim and Hassan 2011). Similarly, unbound hemoglobin and heme released during intravascular hemolysis are highly inflammatory, cytotoxic and contributory towards vascular and tissue damage in SCD (Merle et al. 2019; Thomas et al. 2019).

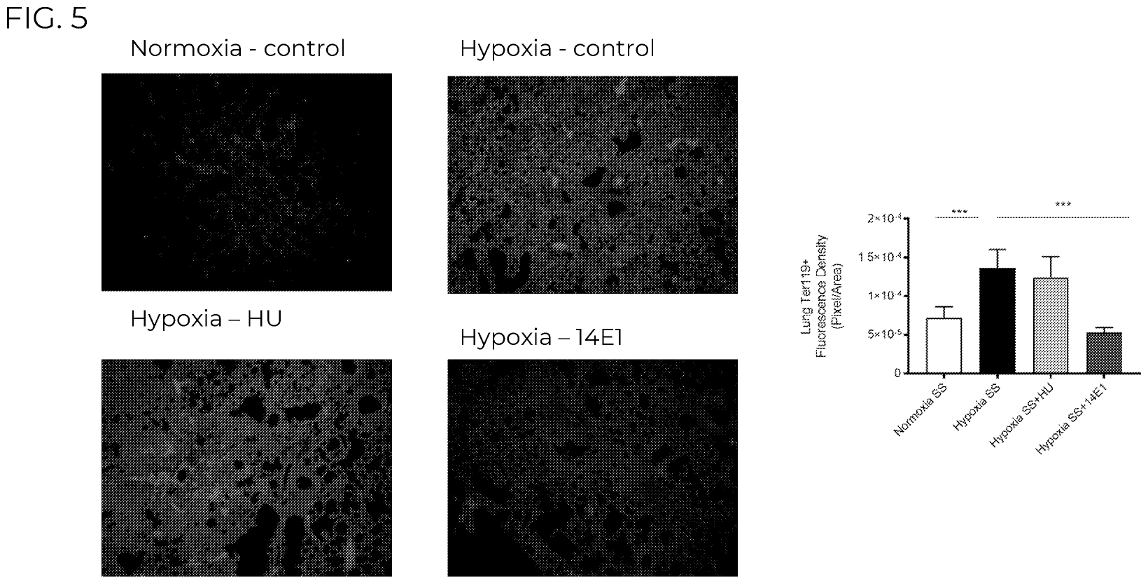

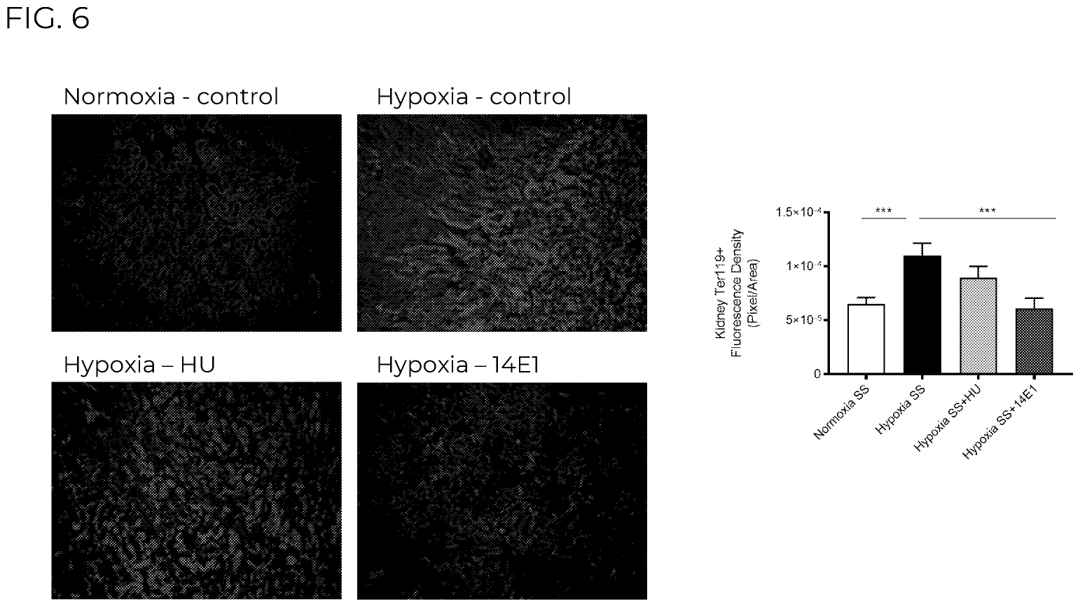

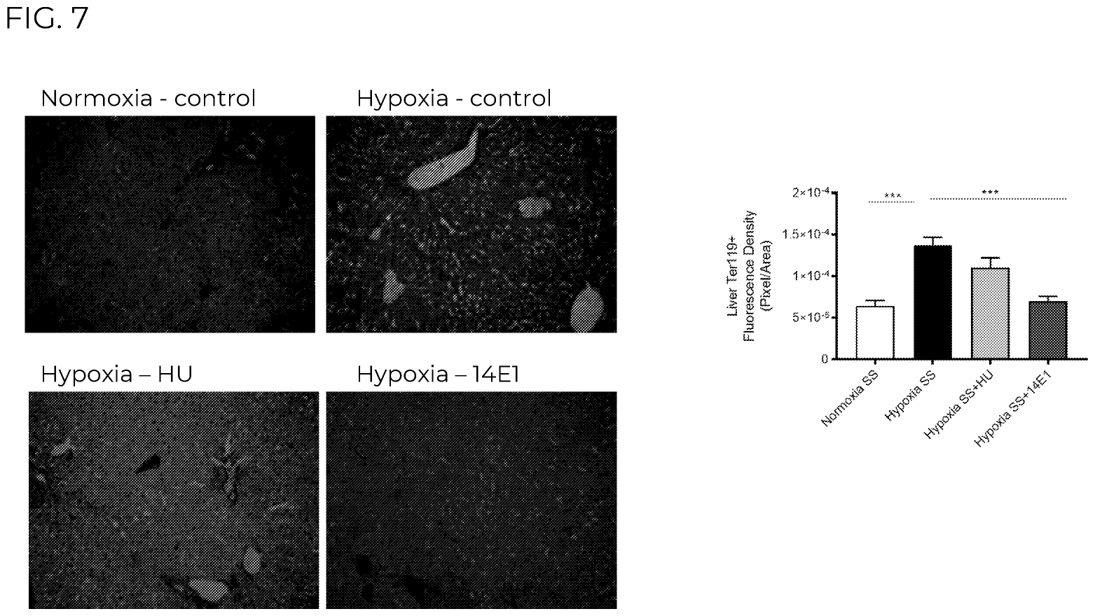

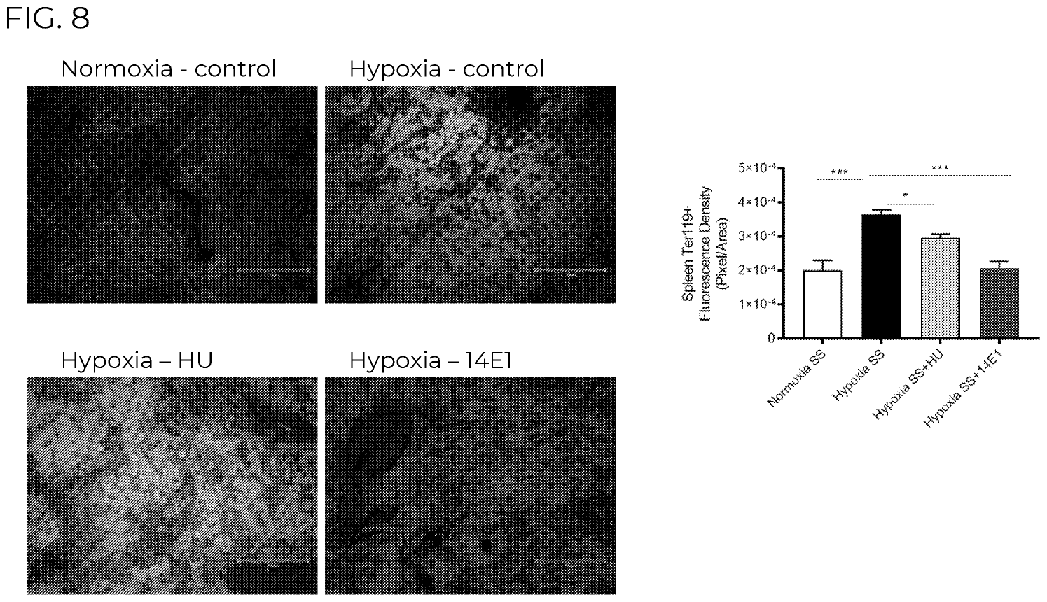

Next, levels of in situ vaso-occlusion was visualized and quantified by immunofluorescence (IF) staining of RBCs (Ter-119). An assay was carried out to measure the extent of clogging the vessels in vital organs such as lung (FIG. 5) kidney (FIG. 6), liver (FIG. 7), and spleen (FIG. 8). Exposure of SCD mice to a hypoxic condition markedly increased the intensity of vaso-occlusion in the lung as well as liver. Pre-treatment with anti-properdin MAb (14E1) effectively reduced the level of vaso-occlusion in comparison to control (treatment with phosphate buffered saline; PBS). Results from representative experiments are presented in the photomicrographs and further summarized in the bar charts of FIGS. 5-8.

Amelioration of vaso-occlusion in the lung is particularly relevant as vaso-occlusion in the lung is an underlying cause of acute chest syndrome (ACS) (Jain, Bakshi, and Krishnamurti 2017). ACS is associated with a high risk of sickle cell-related mortality and morbidity in children, including prolonged hospitalization. More than half of all children with homozygous SCD (HbSS) experience at least one episode of ACS in the first decade of life (Gill et al. 1995). Recurrent episodes may herald the onset of debilitating chronic lung disease (Powars et al. 1988). Therefore, significant amelioration of vaso-occlusion in the lung by anti-properdin provides clear rationale for anti-complement therapy for the treatment of SCD.

Anti-properdin pretreatment also have profound impacts in amelioration of vaso-occlusion in the liver (FIG. 7). The data show that hypoxia-induced liver VOC in the in vivo mouse model is significantly reduced when mice are treated with anti-properdin antibody.

These data establish that an anti-complement antibody, such as an anti-properdin antibody, protects animals inflicted with sickle cell disease against liver VOC. Acute VOC in the liver is an underlying cause of severe abdominal pain and liver dysfunction (Ebert, Nagar, and Hagspiel 2010). In patients admitted for acute vaso-occlusive crisis (severe pain in chest, abdomen, and joints), the liver is involved in about 39% of cases (Koskinas et al. 2007). These patients present with abdominal meteorism, right upper quadrant pain, or acute painful hepatomegaly (Koskinas et al. 2007). Therefore, the data presented here further supports treatment of SCD, BT, and sickle BT patients with properdin antagonists such as anti-properdin antibodies.

Collectively, the data establishes that sickle RBCs undergo SCD pathology including hemolysis and vaso-occlusion through complement activation. Furthermore, using anti-complement therapy, particularly, via therapy with properdin antagonists substantially improves SCD disease phenotype, at both tissue (e.g., lung, kidney, liver, or spleen) as well as cellular level.

### Example 2. Efficacy of Inhibition of Complement Activation in Heme-Induced Vaso-Occlusion Crisis

This study used male Townes S/S mice on a 129/B6 mixed genetic background (Wu et al. 2006). In Townes S/S mice, mouse α- and β-globin gene loci are deleted and replaced by human α and ^{A}γβ^{S}globins. When carrying two copies of the β^{S} allele (hα/hα::β^{S}/β^{S}), mice develop a human sickle disease phenotype with sickle-shaped red blood cells (RBCs) seen in blood smears. Breeding pairs were obtained from the Jackson Laboratories. The animals were housed under conventional conditions at the Animal Care Facility at Imagine Institute.

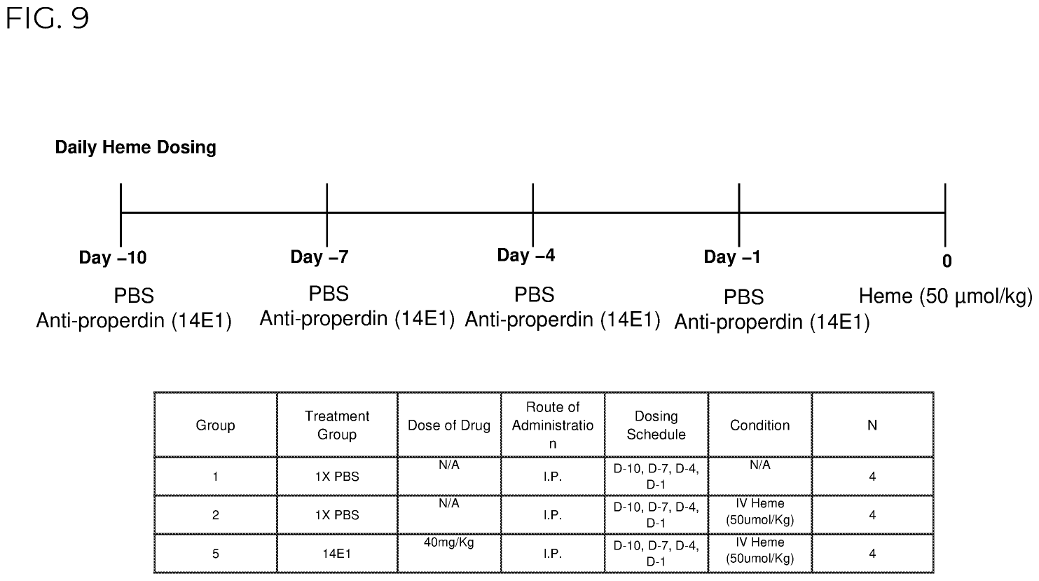

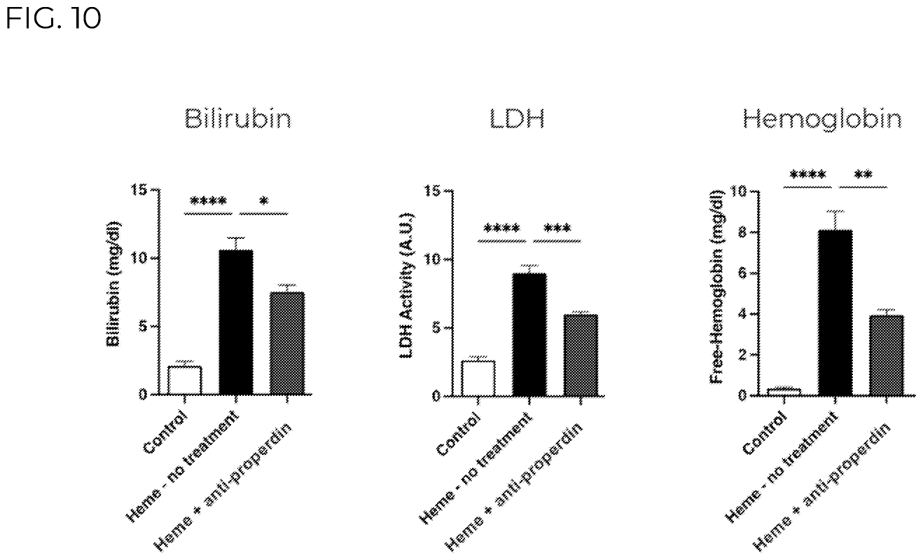

To demonstrate the efficacy of inhibition of complement activation in VOC, Townes SS mice were divided into five groups and prophylactically treated with PBS (vehicle), or anti-properdin mAb (14E1) four times from ten days before heme treatment (FIG. 9). The animals were exposed to 50 μmol/Kg of heme for three hours after which the animals were sacrificed (FIG. 9). In one of the vehicle-treated group, animals were not exposed to heme and served as a baseline (FIG. 9). Upon euthanasia, blood samples and critical organs were harvested from the animals to measure the level of complement deposition on RBCs, intravascular hemolysis, and the severity of vaso-occlusions (FIG. 10). Mice were phlebotomized by retro-orbital bleeding using a capillary tube internally coated with heparin/EDTA anticoagulant. Mice were euthanized by cervical dislocation and perfused with 1 mL of saline solution through the left ventricle. Lung, liver, kidney and spleen were collected and weighed.

Plasma heme was measured using Hemin Assay Kit (Sigma-Aldrich reference MAK036), determined by a coupled enzyme reaction, which resulted in a colorimetric (570 nm) product, proportional to the hemin present in plasma. Plasma was diluted 1:4 with hemin assay buffer to a final volume of 50 μL. The reaction mix was prepared in duplicate in the following order: 3 μL enzyme mix, 2 μL hemin substrate, 43 μL hemin assay buffer and 2 μL hemin probe. Hemoproteins present in the plasma can generate a background signal, so to control for this variable, a blank was prepared for each sample by omitting the enzyme from reaction mix. The reaction mix was added to samples in a 96 well-plate, homogenized using a horizontal shaker and incubated 30 minutes at room temperature, protected from light. A hemin standard solution was prepared in the 96-well plate by diluting the hemin standard provided in the kit. Absorbance was measured at 570 nm in kinetic mode using an Infinite F200 Pro multimode plate reader (Tecan). The background signal was removed by subtracting the blank sample value from each sample reading to obtain the corrected measurement. The hemin concentration was determined by plotting the corrected measurement to a standard curve.

The level of intravascular hemolysis was determined by multiple measures including total bilirubin, plasma lactate dehydrogenase (LDH) activity, and free hemoglobin. Exposure of the SCD animals to heme triggered intravascular hemolysis, which is effectively prevented by pretreatment with anti-properdin antibodies (FIG. 10).

Plasma bilirubin was measured using a Bilirubin Assay Kit (Sigma-Aldrich reference MAK126), based on the Jendrassik-Grof method. This method was based on the reaction of bilirubin with diazotized sulfanilic acid, resulting in a colorimetric product measured at 530 nm, proportionate to the bilirubin present in the sample. Total bilirubin was determined by the addition of Reagent C containing caffeine benzoate which splits bilirubin from the unconjugated bilirubin-protein complex. Plasma was diluted 1:2 with PBS to a final volume of 50 μL. Work reagent was prepared in the following order: 50 μL reagent A, 20 μL reagent B and 130 μL reagent C. A blank was prepared for each sample by omitting the reagents B and C from the reaction mix (replaced by saline solution). The reaction mix was added to samples in a 96 well-plate, homogenized using a horizontal shaker and incubated 10 minutes at room temperature, protected from light. Absorbance was measured at 530 nm using an Infinite F200 Pro multimode plate reader (Tecan). Background was removed by subtracting the blank sample value from each sample reading to obtain the corrected measurement. Bilirubin concentration was determined by the following equation: [(Sample−Blank)/(Calibrator−Water)]×5 mg/dL.

Whole blood was collected on K2 EDTA tubes (Melet Schloesing Laboratoires). Cells were removed from plasma by centrifugation for 15 minutes at 2,000×g using a refrigerated centrifuge. This step also depletes platelets in the plasma sample. Plasma was apportioned into 50 μL aliquots and stored at −80° C.

Plasma LDH was measured using a Pierce LDH Cytotoxicity Assay Kit (Thermofisher Scientific reference 88953). Reaction mix was prepared by combining 0.6 mL of assay buffer with 11.4 mL of substrate mix in a 15 mL conical tube. Plasma was diluted 1:2 with PBS to a final volume of 50 μL.

Reaction mix was added to samples in a 96 well-plate, homogenized using a horizontal shaker and incubated 30 minutes at room temperature, protected from light. The reaction was stopped by adding 50 μL of stop solution to each sample. Absorbance was measured at 490 nm and 680 nm using an Infinite F200 Pro multimode plate reader (Tecan). LDH activity was determined as [(LDH 490 nm)−(LDH 680 nm)].

Plasma hemoglobin was measured using Drabkin's Reagent (Sigma-Aldrich reference D5941). This procedure was based on the oxidation of hemoglobin and its derivatives (except sulfhemoglobin) to methemoglobin in the presence of alkaline potassium ferricyanide. Methemoglobin reacts with potassium cyanide to form cyanmethemoglobin, which had maximum absorption at 540 nm. The color intensity measured at 540 nm is proportional to the total hemoglobin concentration. Plasma was transferred to a 96 well-plate (20 μL for each sample). Drabkin's solution was prepared by reconstituting one vial of the Drabkin's reagent with 1,000 mL of water and 0.5 mL of 30% Brij L23 Solution, (Sigma Catalog Number B4184). Drabkin's solution (180 μL) was added to samples in a 96 well-plate, homogenized using a horizontal shaker and incubated 15 minutes at room temperature, protected from light. Hemoglobin calibration curve was prepared in Drabkin's solution. Absorbance was measured at 540 nm using an Infinite F200 Pro multimode plate reader (Tecan). Background was removed by subtracting the blank sample value from each sample reading to obtain the corrected measurement. Hemoglobin concentration was determined by plotting the corrected measurement to a calibration curve.

Blood (45 μL) was incubated with 5 μL of mouse FcR Blocking Reagent (Miltenyi Biotec reference 130-092-575) for 10 minutes and diluted 1:2 with 50 μL of cell staining buffer (Biolegend reference 420201). Samples were then stained with antibodies against Ter-119 Pacific Blue (Biolegend reference 116232; 1/100 dilution), mouse TfR1/CD71 PerCP/Cy5.5 (Biolegend reference 113816; 1/100 dilution), C5b9-FITC (Santa Cruz Biotechnologies reference sc-66190 FITC; 1/20 dilution) or C3-FITC (Cedarlane reference CL7631F; 1/50 dilution). Dead cells were excluded by Live-Dead (eBioscience).

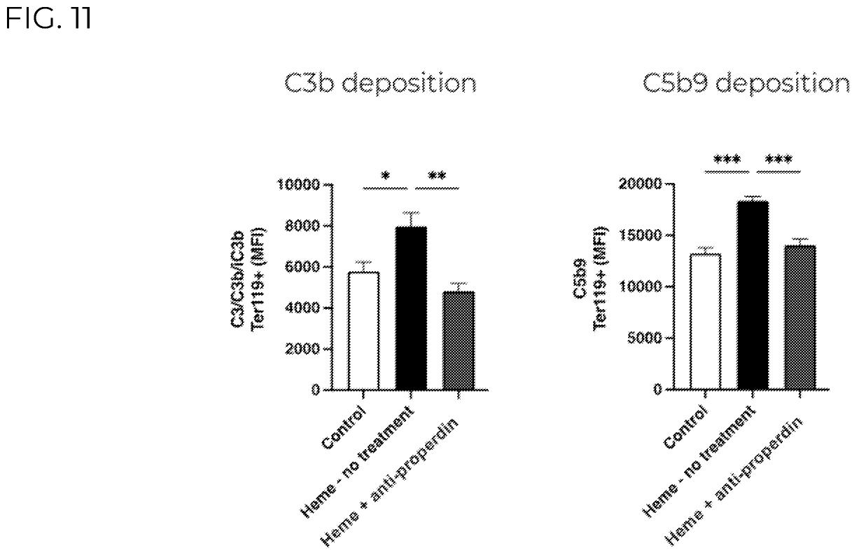

Cells were further analyzed by flow cytometry (Gallios Beckman Coulter) using FlowJo software (Tree Star). Flow cytometry-based SS RBC analyses revealed marked increase in both C5b9 and C3 deposition on SS RBCs upon exposure to heme (FIG. 11). Pretreatment with anti-properdin nearly completely prevented the increase in C5b9 deposition on SS RBCs (FIG. 11). As C5b9 staining represents potential membrane attack complex (MAC) formation, prevention of C5b9 deposition is expected to reduce complement-mediated intravascular hemolysis (FIG. 11). Next, C3 deposition was then measured on SS RBCs. The increase in C3 deposition upon exposure to heme was markedly reduced by an anti-properdin antibody (FIG. 11).

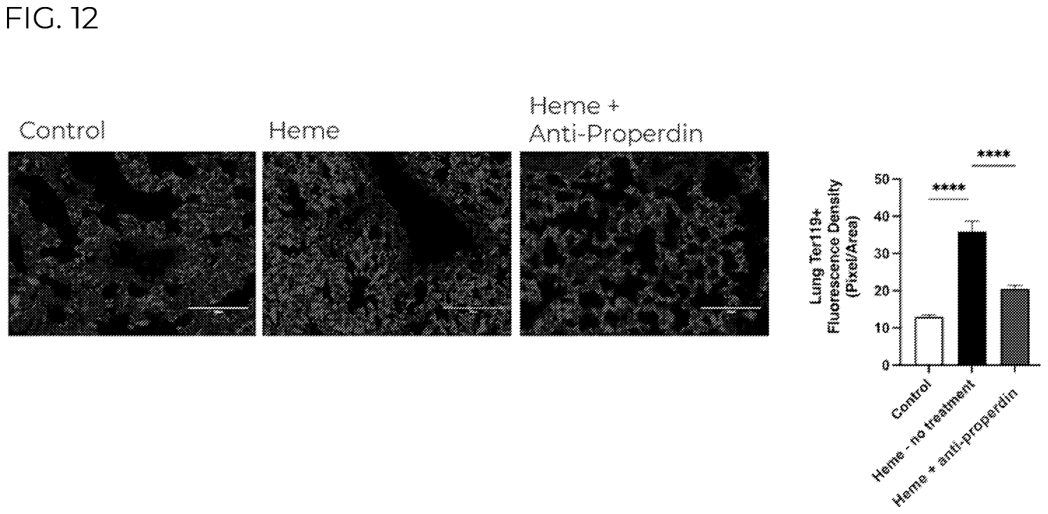

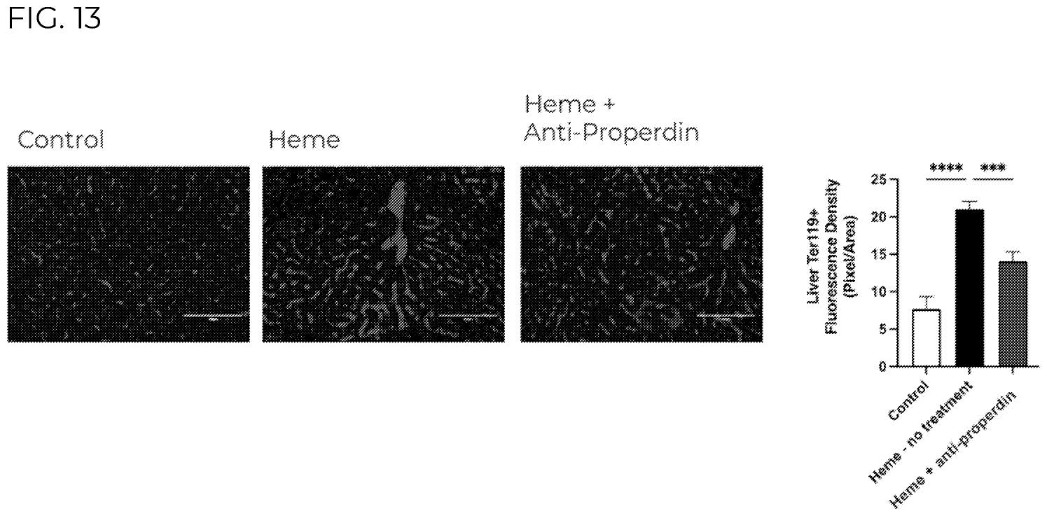

Paraffin-embedded lung, spleen, liver or kidney sections (5 μm) were processed for deparaffinization, rehydration and antigen retrieval using a citrate buffer for 20 minutes at 95° C. (Biolegend reference 928502). Samples were delimited with a PAP-pen, blocked 15 minutes with high protein IHC/ICC blocking buffer (eBioscience reference 00-4952-54) and then incubated 1 hour with primary antibodies against Ter-119, a marker for vessel-trapped RBCs, coupled to alexa fluor-488 (Biolegend reference 116215; 1/100 dilution). Slides were washed thoroughly with TBS Tween-20 0.05% for 3×10 minutes and mounted with prolong diamond antifade mountant with DAPI (ThermoFischer Scientifc reference P36962). Images were acquired on an EVOS M5000 Imaging System (ThermoFisher Scientific) at magnification×200 and positive pixels per area were analyzed using ImageJ software. The intensity of vaso-occlusion was visualized and quantified by immunofluorecence (IF) staining of RBCs (Ter-119) clogging the vessels in vital organs including the lung and liver (FIG. 12 and FIG. 13, respectively). Exposure of the SCD mice to heme markedly increased the intensity of vaso-occlusion in the lung and liver. Pretreatment with anti-properdin monoclonal antibodies effectively reduced the level of vaso-occlusion in a statistically significant manner in comparison to PBS treatment.

Statistical analyses studies were performed using a one-way analysis of variance (ANOVA) test followed by a Tukey's test (multiple comparison test) or Kruskal-Wallis test (non-parametric) for analysis of treatment effect versus controls. All statistical analyses were derived using GraphPad software (v6.00, San Diego, California, USA). Statistical significance to reject the null hypothesis was identified at the p<0.05 level. For illustrative purposes, significance levels of P<0.01 and P<0.005 were also noted.

### Example 3: Complement Induced Deposition Assay for C3 and C5b-9

### Induction of Complement Deposition on SS-RBCs by Heme and Assessment of Anti-Properdin Blockade

RBCs and serum from SCD patients homozygous for the mutation in the hemoglobin gene (SS) were obtained from BioIVT (cat HUMANRBCALSUZN and HMRBC-SCA respectively) and from Sanguine Biosciences (Study #24348). Gelatin Veronal Buffer (GVB) was obtained from Boston Bioproducts (cat. IBB-300X). Mg-EGTA (cat. B106), C8 depleted normal human serum (cat. A325) and normal human serum (cat. NHS) were obtained from Complement Technology. PBS was obtained from Corning, cat. 21-031-CV. Porcine heme (Sigma, cat. 51280) was used at various concentrations (50-800 uM) to amplify complement activation and induce deposition on human cells.

All centrifugations were performed at 440×g for 5 min at 4° C. and supernatants aspirated with multi-channel pipets to avoid disturbing the loose RBC pellets.

### Identification of Appropriate Concentration of Heme for Use in Complement Inhibition Assays

Patient SS-RBCs were washed three times in PBS, resuspended in GVB, 5 mM Mg-EGTA and re-distributed to sterile V-bottom 96 well plates at a concentration of 2×10⁶ cells/well. Autologous serum was added to 20% final concentration. Heme was used at 0, 100, 200, 400, and 800 μM. Following incubation for 20-30 minutes at 37° C. 5% CO₂, PBS containing 10% EDTA (Corning, cat. 46-030-CI) was added to stop complement activation. RBCs were washed and stained with antibody to iC3b as detailed below.

### AP Blockade of Complement Deposition on SS-RBCs Induced by Heme

Patient SS-RBCs were washed two or three times in PBS. To induce complement deposition, RBCs were resuspended in GVB, 5 mM Mg-EGTA (assay buffer) at 5×10⁷ cells/mL and 30 μL added to sterile V-96 wells. Normal human serum was added to 20% final concentration. Complement inhibitors were diluted in assay buffer at 5× working stock of 3.125 μM and 10 μL added to wells containing cells. Porcine heme was added to 400 μM and the cells incubated for 20-30 minutes at 37° C., 5% CO₂. Complement activation was stopped by the addition of 150 μL/well PBS containing 10 mM EDTA. Cells were centrifuged and washed once with 200 μL PBS and stained for iC3b and C5b-9 deposition below.

Flow Cytometric Analysis of iC3b and C5b-9 Deposition on the Surface of SS-RBCs

Cells were resuspended in 50 μL per well iC3b (Quidel, cat. A209) or C5b-9 antibody (Quidel, cat. A239) diluted to 4 μg/mL in PBS and incubated for 20-30 min at 4° C., staining for flow cytometry was performed in sheath fluid. Cells were washed twice with 150-200 μL PBS, resuspended in 50 μL goat anti-mouse IgG (H+L)-AF488 (Invitrogen cat. A11029) diluted to 4 μg/mL in PBS and incubated for 20-30 min at 4° C. In some experiments, goat anti-mouse IgG2b AF488 was used at 4 μg/mL (Invitrogen, cat. A21141). Cells were washed twice with 150-200 μL PBS and acquired on the LSR Fortessa for flow cytometric analysis.

### Results

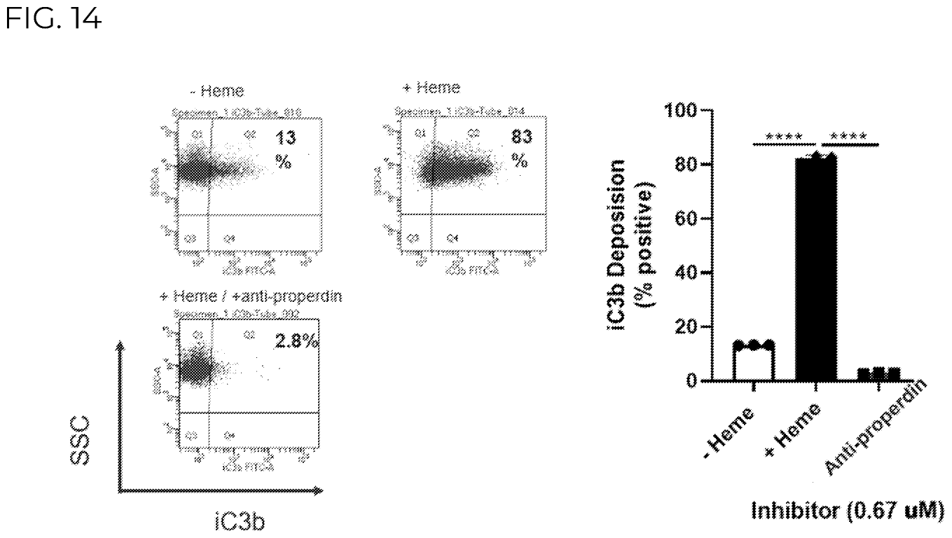

FIG. 14 shows flow cytometry-based data on heme-induced complement deposition on sickle RBCs and the effect of anti-properdin antibody treatment. SCD red blood cells were exposed to 400 μM heme in the presence of 20% normal human serum (NHS). On the left are scatterplots showing iC3b deposition under various conditions, including normal, heme, and heme+anti-properdin antibody pretreatment. On the right is a bar graph quantifying the iC3b levels. For the data shown in FIG. 14, significance levels of ****P<0.0001 and **P<0.01 were noted. FIG. 14 shows that heme-triggered complement deposition on SCD RBCs was blocked in the presence of anti-properdin antibody by >95% for iC3b and by >85% for C5b-9.

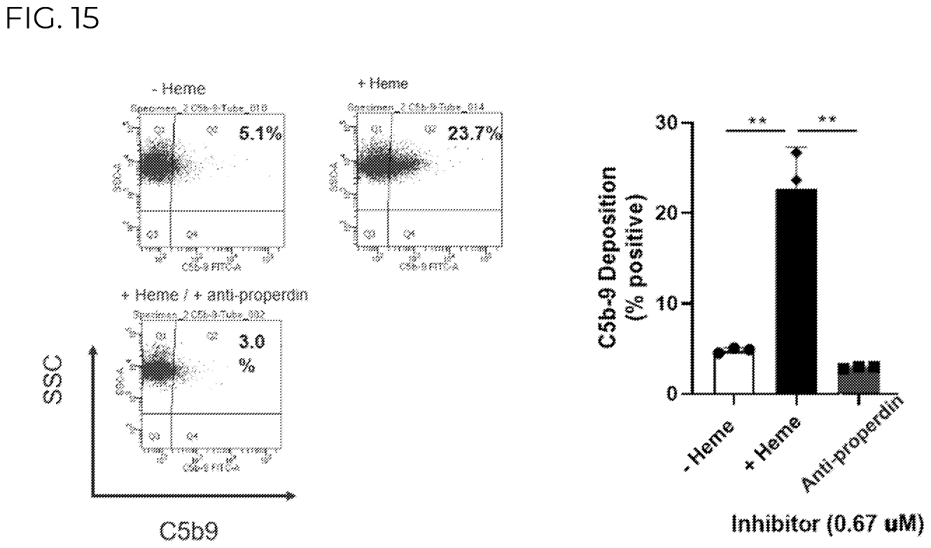

FIG. 15 shows flow cytometry-based data on heme-induced complement deposition on sickle RBCs and the effect of anti-properdin antibody treatment. SCD red blood cells were exposed to 400 μM heme in the presence of 20% normal human serum (NHS). On the left are scatterplots showing C5b9 deposition under various conditions, including normal, heme, and heme+anti-properdin. On the right is a bar graph quantifying the C5b9 levels. For the data shown in FIG. 15, significance levels of **P<0.01 were noted. FIG. 15 shows heme-triggered complement deposition on SCD RBCs was blocked in the presence of anti-properdin antibodies by >95% for iC3b and by >85% for C5b-9.

As shown in FIG. 14 and FIG. 15, heme triggered significant levels of iC3b and C5b-9 deposition on red blood cells from sickle cell patients. Significant levels of P<0.0001 and <0.01 for iC3b and C5b-9 respectively as determined by students t test. Heme-triggered complement deposition on SCD RBCs was blocked in the presence of anti-properdin monoclonal antibody by >95% for iC3b and by >85% for C5b-9 (P<0.0001 and <0.01 respectively).

### Example 4: AP Inhibitors Block Heme Induced Complement Deposition on HMEC-1 Cells

The endothelial cell line HMEC-1 was purchased from ATCC (CRL 3243) and expanded and banked at AcCellerate (Cat. CBA02, lot 92-190318FG01). This is a dermal microvascular endothelial cell line. Cells were used in experiments at passage<5.

All centrifugation steps were performed for 5-7 min at 300 g at room temperature (RT). HMEC-1 cells were seeded into 6 well plates at 1.5×10⁵ cells per well in medium (Endothelial cell growth medium MV2, Promocell, cat. 22022) and allowed to reach confluency (72 hrs). Normal human serum (Complement Technologies, cat. NHS) was spiked with 1 uM inhibitor, diluted to 20% using Live cell imaging solution (LCIS) (Invitrogen, cat A1429DJ) containing 5-10 mM MgEGTA and added to HMEC-1 cultures in place of the medium. Alternatively, LCIS without MgEGTA was used as the test buffer. Heme was added to 400 μM, mixed and incubated for 20-30 min at 37° C. Cells were rinsed twice with 2 mLs PBS (Corning, cat. 21-031-CV) and detached with PBS containing 10 mM EDTA (Corning, cat. 46-034-CI). Cells were centrifuged, pellets resuspended in 400 μL sheath fluid (BD Biosciences, cat. 342003) and transferred to V-bottom 96 well plates in duplicate. After centrifugation, pellets were resuspended in 50 μL per well sheath fluid containing either iC3b or C5b-9 antibody diluted to 4 μg/mL. Following several washes, the cells were incubated with 50 μL of goat anti mouse IgG (H+L) AF 488 diluted to 4 μg/mL in sheath fluid for 30 min at 4° C. Following several washes, cells were acquired on the LSR Fortessa for flow cytometry analysis.

### Results

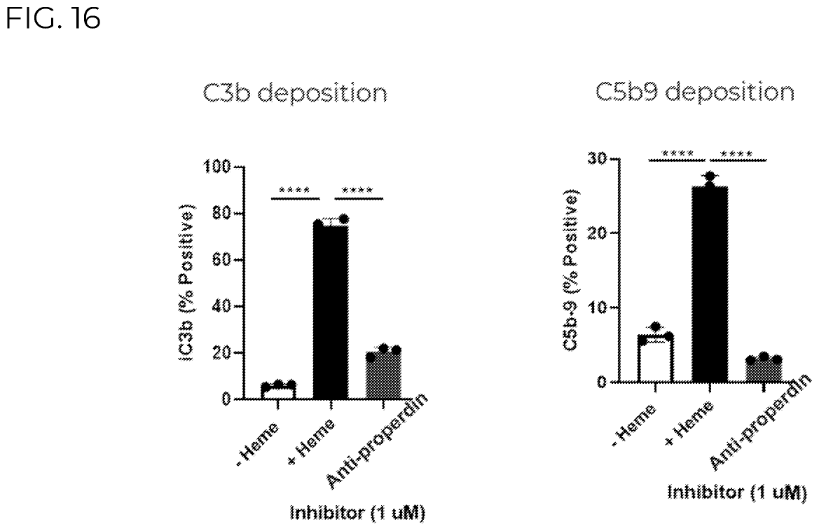

FIG. 16 shows bar charts showing flow cytometry-based analyses of heme-induced complement fragment deposition on endothelial cells exposed to heme and the effect of anti-properdin antibodies on complement deposition. Shown are changes in complement fragment levels, from left to right, normal, heme, and heme+anti-properdin pretreatment. The left-hand panel shows C3/C3b/iC3b deposition and the right-hand panel shows C5b9 deposition. For the data shown in FIG. 16, significance levels of P<0.0001 were noted.

As shown in FIG. 16, heme potently triggered deposition of iC3b and C5b-9 on HMEC-1 cells (P<0.0001 for both). In the presence of anti-properdin antibodies, deposition on HMEC-1 was blocked by >70% for iC3b and >85% for C5b-9 (P<0.0001 for both).

### REFERENCES

The following references are incorporated by reference herein in their entirety:
Ballas et al. Hyperhemolysis during the Evolution of Uncomplicated Acute Painful Episodes in Patients with Sickle Cell Anemia. Transfusion 46(1): 105-110, 2006.
Blatt et al. Properdin: A Tightly Regulated Critical Inflammatory Modulator. Immunological Reviews 274(1): 172-190, 2016.
Ebert et al. Gastrointestinal and Hepatic Complications of Sickle Cell Disease. Clinical Gastroenterology and Hepatology 8(6): 483-489, 2010.
Frimat et al. Complement Activation by Heme as a Secondary Hit for Atypical Hemolytic Uremic Syndrome. Blood 122(2): 282-292, 2013.
Gill et al. Clinical Events in the First Decade in a Cohort of Infants with Sickle Cell Disease. Cooperative Study of Sickle Cell Disease. Blood 86(2): 776-783, 1995.
Gullipalli et al. Antibody Inhibition of Properdin Prevents Complement-Mediated Intravascular and Extravascular Hemolysis. Journal of Immunology (Baltimore, Md.: 1950) 201(3): 1021-1029, 2018.
Jain et al. Acute Chest Syndrome in Children with Sickle Cell Disease. Pediatric Allergy, Immunology, and Pulmonology 30(4): 191-201, 2017.
Kato et al. Lactate Dehydrogenase as a Biomarker of Hemolysis-Associated Nitric Oxide Resistance, Priapism, Leg Ulceration, Pulmonary Hypertension, and Death in Patients with Sickle Cell Disease. Blood 107(6): 2279-2285, 2006.
Koskinas et al. Liver Involvement in Acute Vaso-Occlusive Crisis of Sickle Cell Disease: Prevalence and Predisposing Factors. Scandinavian Journal of Gastroenterology 42(4): 499-507, 2007.
Lombardi et al. Factor H Interferes with the Adhesion of Sickle Red Cells to Vascular Endothelium: A Novel Disease-Modulating Molecule. Haematologica 104(5): 919-928, 2019.
Merle et al. Complement Activation during Intravascular Hemolysis: Implication for Sickle Cell Disease and Hemolytic Transfusion Reactions. Transfusion Clinique Et Biologique: Journal De La Societe Francaise De Transfusion Sanguine 26(2): 116-124, 2019.
Merle et al. Intravascular Hemolysis Activates Complement via Cell-Free Heme and Heme-Loaded Microvesicles. JCI Insight 3(12), 2018.
Miwa et al. Blocking Properdin, the Alternative Pathway, and Anaphylatoxin Receptors Ameliorates Renal Ischemia-Reperfusion Injury in Decay-Accelerating Factor and CD59 Double-Knockout Mice. Journal of Immunology (Baltimore, Md.: 1950) 190(7): 3552-3559, 2013.
Najim et al. Lactate Dehydrogenase and Severity of Pain in Children with Sickle Cell Disease. Acta Haematologica 126(3): 157-162, 2011.
Powars et al. Sickle Cell Chronic Lung Disease: Prior Morbidity and the Risk of Pulmonary Failure. Medicine 67(1): 66-76, 1988.
Stankovic et al. Lactate Dehydrogenase in Sickle Cell Disease. Clinica Chimica Acta; International Journal of Clinical Chemistry 458: 99-102, 2016.
Test et al. Defective Regulation of Complement by the Sickle Erythrocyte: Evidence for a Defect in Control of Membrane Attack Complex Formation. Blood 83(3): 842-852, 1994.
Thomas et al. Complement Component C5 and TLR Molecule CD14 Mediate Heme-Induced Thromboinflammation in Human Blood. Journal of Immunology (Baltimore, Md.: 1950) 203(6): 1571-1578, 2019.
Ueda et al. Blocking Properdin Prevents Complement-Mediated Hemolytic Uremic Syndrome and Systemic Thrombophilia. Journal of the American Society of Nephrology: JASN 29(7): 1928-1937, 2018.
Vercellotti et al. Critical Role of C5a in Sickle Cell Disease. American Journal of Hematology 94(3): 327-337, 2019.
Wang et al. Activation of the Alternative Complement Pathway by Exposure of Phosphatidylethanolamine and Phosphatidylserine on Erythrocytes from Sickle Cell Disease Patients. The Journal of Clinical Investigation 92(3): 1326-1335, 1993.
Wu et al. Correction of Sickle Cell Disease by Homologous Recombination in Embryonic Stem Cells. Blood 108(4): 1183-1188, 2006.
Zhou et al. Antibody Directs Properdin-Dependent Activation of the Complement Alternative Pathway in a Mouse Model of Abdominal Aortic Aneurysm. Proceedings of the National Academy of Sciences of the United States of America 109(7): E415-422, 2012.

### Other Embodiments

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries (e.g., PUBMED, NCBI, FDA Drug, or UNIPROT accession numbers), and other references mentioned herein are incorporated by reference in their entirety. In case of conflict, the present specification, including definitions, will control.

While particular embodiments of the present disclosure have been illustrated and described, it would be obvious to those skilled in the art that various other changes and modifications may be made without departing from the spirit and scope of the disclosure. It is therefore intended to cover in the appended claims all such changes and modifications that are within the scope of this disclosure.

What is claimed is:

1. A method for treating sickle cell disease (SCD) in a subject, comprising administering to the subject an effective amount of a composition comprising a complement alternative pathway inhibitor.

2. A method for treating β-thalassemia (BT) in a subject, comprising administering to the subject an effective amount of a composition comprising a complement alternative pathway inhibitor.

3. A method for treating sickle cell BT in a subject, comprising administering to the subject an effective amount of a composition comprising a complement alternative pathway inhibitor.

4. The method of any one of claims 1-3, wherein the complement alternative pathway inhibitor is selected from the group consisting of an antibody or an antigen-binding fragment thereof, a peptide, a small molecule, a nucleic acid molecule, and an aptamer.

5. The method of any one of claims 1-3, wherein the complement alternative pathway inhibitor is a properdin inhibitor.

6. The method of claim 5, wherein the properdin inhibitor is an anti-properdin antibody or antigen-binding fragment thereof.

7. The method of claim 6, wherein the anti-properdin antibody or antigen-binding fragment thereof comprises:
(a) CDR-H1 (SEQ ID NO: 2), CDR-H2 (SEQ ID NO: 3), and CDR-H3 (SEQ ID NO: 4).

8. The method of claim 6, wherein the anti-properdin antibody or antigen-binding fragment thereof comprises:
(a) the anti-FP V_{HH} component of SEQ ID NO: 6;
(b) the sequence of SEQ ID NO: 6;
(c) the V_{HH} of SEQ ID NO: 31;
(d) the V_{HH} of SEQ ID NO: 32;
(e) the V_{HH} of SEQ ID NO: 33; or
(f) the V_{HH} of SEQ ID NO: 34.

9. The method of claim 4, wherein the peptide inhibits complement factor C3.

10. The method of claim 4, wherein the small molecule is a complement factor D inhibitor.

11. The method of claim 4, wherein the nucleic acid molecule is selected from the group consisting of small interfering RNA, short hairpin RNA, micro RNA and antisense oligonucleotide.

12. The method of claim 11, wherein the nucleic acid molecule is complementary to a portion of an endogenous nucleic acid sequence encoding complement C3.

13. The method of any one of claims 1-12, wherein the composition comprises the complement inhibitor and a pharmaceutically acceptable carrier.

14. The method of claim one of claims 1-13, wherein the method reduces intravascular hemolysis in the subject.

15. The method of any one of claims 1 and 4-14, wherein the SCD comprises hemolytic anemia or an acute vaso-occlusion (VOC) event.

16. The method of claim 15, wherein the subject presents with abdominal meteorism, right upper quadrant pain, or acute painful hepatomegaly.

17. The method of claim 15, wherein the VOC event is a lung VOC and/or a liver VOC.

18. The method of claim 17, wherein:
(a) the lung VOC manifests as acute chest syndrome (ACS) and/or chronic lung disease; and/or
(b) the liver VOC manifests as severe abdominal pain and/or liver dysfunction.

19. The method of any one of claims 1-18, wherein the subject is a human patient diagnosed as having SCD, BT, or sickle cell BT.

20. The method of claim 19, wherein the human patient is under 18 years of age.

21. The method of claim 1, wherein the subject having SCD is diagnosed as having a mutation in the p globin gene.

22. The method of claim 21, wherein the mutation in the β globin gene is a single nucleotide mutation in the β globin gene.

23. The method of claim 22, wherein the single nucleotide mutation in the β globin gene results in a glutamic acid substitution by valine at position 6, relative to SEQ ID NO: 1.

24. The method of claim 1, wherein the SCD comprises complement deposition in red blood cells (RBC).

25. The method of claim 24, wherein the SCD comprises C5b9 deposition in RBC.

26. The method of claim 1, wherein the SCD comprises intravascular hemolysis (IVH).

27. The method of claim 26, wherein IVH is characterized by an increase in at least one marker comprising lactate dehydrogenase (LDH), bilirubin, free hemoglobin, and free heme.

28. The method of any one of claims 1-3, wherein upon administration of the complement alternative pathway inhibitor to the subject, the subject exhibits a reduction in a SCD, a BT or a sickle cell BT phenotype.

29. The method of claim 28, wherein the SCD phenotype comprises increased inflammation or cytotoxicity leading to vascular tissue damage; enhanced pain triggered by VOC events; or increases in mortality or morbidity of SCD patients.

30. The method of any one of claims 1-29, wherein the composition is administered intravenously.

31. A method for improving viability or reducing death of cells under hypoxic conditions comprising contacting the cells with an effective amount of a composition comprising a complement alternative pathway inhibitor.

32. The method of claim 31, wherein the cells are contacted in vivo.

33. The method of claim 31 or 32, wherein the cells are sickle cells.

34. The method of any one of claims 31-33, wherein the complement alternative pathway inhibitor is a properdin inhibitor.

35. The method of claim 34, wherein the properdin inhibitor is selected from the group consisting of an anti-properdin antibody or a bi-specific antibody comprising at least one moiety that binds to properdin.

36. The method of any one of the aforementioned claims wherein SCD is characterized by a feature selected from:
(a) increased deposition of complement C3 and/or C5b9 in affected cells (e.g., RBCs), especially under a trigger (e.g., hypoxia);
(b) increased neovascular hemolysis, especially under a trigger (e.g., hypoxia), wherein increased hemolysis is characterized by increases in plasma LDH activity/levels, free heme and/or free hemoglobin levels, and/or total bilirubin levels; or
(c) increased severity of VOC, especially under a trigger (e.g., hypoxia).

37. The method of any one of the aforementioned claims, wherein treatment with a complement inhibitor results in an outcome selected from:
(a) inhibition or reversal of complement fragment deposition of C3 and C5b9 in RBCs of the subject with SCD, e.g., under hypoxic conditions;
(b) attenuation or reversal in the level of intravascular hemolysis under hypoxic conditions (as measured increases in plasma LDH activity/levels, free heme and/or free hemoglobin levels, and/or total bilirubin levels); or
(c) reduction or reversal in vaso-occlusion in the vessels of vital organs such as lung, kidney, liver and spleen of the subject with SCD.

38. The method of claim 37, wherein treatment with a complement inhibitor results in an improvement in an at least one outcome from (a)-(c) compared to treatment of the subject with hydroxyurea.

39. A composition comprising a complement alternative pathway inhibitor for use in treating SCD or a symptom related thereto in a subject, particularly for improving viability of blood cells harboring one or mutations that renders them susceptible to hypoxia or low oxygen tension, e.g., mutation of normal hemoglobin A (α2ß2) to hemoglobin S (α2ß 6 Val2) or mutation in the β-globulin gene of RBC.

40. The composition for use of claim 39, wherein the complement alternative pathway inhibitor is a properdin inhibitor.

41. The composition for use of claim 40, wherein the properdin inhibitor is selected from the group consisting of an anti-properdin antibody or a bi-specific antibody comprising at least one moiety that binds to properdin.

42. The composition for use of claim 41, wherein the nucleic acid molecule is selected from the group consisting of small interfering RNA, short hairpin RNA, micro RNA and antisense oligonucleotide.

43. A composition comprising a complement alternative pathway inhibitor for use in improving viability or reducing death of cells under hypoxic conditions.

44. The composition of claim 43, wherein the complement alternative pathway inhibitor is a properdin inhibitor.

45. The composition of claim 44, wherein the properdin inhibitor is selected from the group comprising an anti-properdin antibody or a bi-specific antibody comprising at least one moiety that binds to properdin.

46. The composition of claim 45, wherein the complement alternative pathway inhibitor is a nucleic acid molecule selected from the group consisting of small interfering RNA, short hairpin RNA, micro RNA and antisense oligonucleotide.
